(19) United States
(12) Patent Application Publication
(10) Pub. No.: US 2022/0142834 A1
(43) Pub. Date: May 12, 2022

(54) A SENSING DEVICE, SYSTEM AND METHOD

(71) Applicant: Loop Plus Pty Ltd, New South Wales (AU)

(72) Inventors: Kathryn HAMILTON, Waitara New South Wales (AU); Filip MLEKICKI, Brooklyn, NY (US)

(21) Appl. No.: 17/434,385

(22) PCT Filed: Feb. 28, 2020
(86) PCT No.: PCT/AU2020/050188

§ 371 Date: Aug. 26, 2021

(30) Foreign Application Priority Data
Feb. 28, 2019 (AU) 2019900649

(51) Int. Cl. A61G 5/10; G08B 21/04; G08B 21/18

(52) U.S. Cl. CPC A61G 5/10; A61G 2203/32; G08B 21/182; G08B 21/0461

(57) ABSTRACT

There is provided a sensing device for use with a mobility assistance device, the sensing device comprising: a sensing layer including a plurality of sensors being arranged along at least one plane, a top outer layer and a bottom outer layer that are sealingly arranged to enclose the sensing layer, wherein the sensing device is arranged to locate between the user and the mobility assistance device, and is configured to attach to the mobility assistance device so that the sensing device remains in the same position relative to the mobility assistance device.

### RELATED APPLICATIONS

This application claims priority from Australian Provisional Patent Application No 2019900649, the entire contents of which are incorporated herein by reference.

### TECHNICAL FIELD

The present invention is directed to a device, system and a method for use with a mobility assistance device or any surface, on any plane, that supports the body. Embodiments of the device, system and method are directed to determining and communicating the state of a user using the mobility assistance device and the calibration of the device.

### BACKGROUND

Wheelchair users and less mobile elderly manage a range of co-morbidities throughout their lives that affects their independence and quality of life. In the past, the monitoring of aged care patients and the care of those with mobility issues has proven very challenging. It is important for individuals with reduced mobility to monitor their activity and environment in order to reduce the risk of further medical issues or conditions developing due to their lack of mobility. Some systems rely on self-evaluation monitoring by the individuals themselves. However, this is not effective in cases were the individual is physically or mentally restricted from monitoring their own activity.

For example, people with spinal cord injuries may have reduced sensitivity to physical stimuli below the location of their spinal cord injury. As such, they will be unable to receive feedback from their nervous system below their injury, such that they may not feel a loss of circulation or the development of pressure sores. Some devices are configured to monitor an aspect of the behaviour or movements of an individual with reduced mobility. However, this single aspect or variable, such as pressure, provides limited insight into the everyday behaviours, activities, environmental conditions that can beneficially or detrimentally impact the health of the wheelchair user.

While prior art has acknowledged that compliance to prescribed pressure relieving actions to prevent pressure injury is poor, known devices persist with timed reminders and tallied reliefs as the output of data measurement.

A further issue faced in the art is that many of the current devices may suffer from erroneous data collection when used in everyday life for extended periods, which can lead the user of the device making decisions based on incorrect information about their or another person's health. For example, due to a range of variables, including a user's level of injury, type of mobility device and cushion, the application of a consistent set of parameters for calibrating sensors and processing data for all users will result in inaccuracies or errors being introduced into the data. While many of the aforementioned devices suggest a calibration method to solve this issue, these devices and methods are not designed for everyday use. For example, everyday wheelchair use can include regular removal of the cushion from the wheelchair, disassembling of the wheelchair frame to pack into a care to enable driving, losing calibration as the device is removed and placed back in to the seat. As such, the medical assessments and recommendations made using such devices will likely be wrong when based on inaccurate or incorrect data.

Additionally, devices that do not remain in a consistent fixed position in a mobility assistance device limit the ability to track the state of the user longitudinally to determine functional recovery or regression. User data with such historical continuity can be used to demonstrate efficacy of clinical intervention and when integrated into health systems alongside electronic health records, changes in the state of the user may predict health issues.

A critical gap in known devices is the timely communication of meaningful insights specific to each user to manage a broad range of health risks everyday. Timely insights could inform early interventions including adjustments to seating apparatus, a change in behavioural habits, a prescribed seating regime, therapeutic or medical interventions that all rely on a timely feedback loop to create collaborative continuous care.

The preferred embodiments of the present invention seek to address one or more of these disadvantages, and/or to at least provide a useful alternative.

### SUMMARY OF INVENTION

A sensing device for use with a mobility assistance device, the sensing device comprising: a sensing layer including a plurality of sensors being arranged along at least one plane, a top outer layer and a bottom outer layer that are sealingly arranged to enclose the sensing layer, wherein the sensing device is arranged to locate between the user and the mobility assistance device, and is configured to attach to the mobility assistance device so that the sensing device remains in the same position relative to the mobility assistance device.

In an embodiment, the mobility assistance device is a wheelchair including a seat frame arranged to support a wheelchair seat, where the sensing device is configured to attach to the seat frame and sit on top of the wheelchair seat by means of one or more mechanical devices.

In an embodiment, the mobility assistance device is a wheelchair including a seat frame arranged to support a wheelchair seat, where the sensing device is configured to attach to the seat frame and replace the wheelchair seat by means of one or more mechanical devices.

In an embodiment, the plurality of sensors includes a plurality of pressure sensors, wherein the plurality of pressure sensors are force-sensing resistors or force-sensing capacitors.

In an embodiment, the plurality of sensors further includes at least one an inertial measurement unit and/or at least one strain measurement device.

In an embodiment, the at least one strain measurement device is attached to a sensing layer facing-side of the top outer layer.

In an embodiment, the at least one inertial measurement unit is arranged in the sensing layer and located proximate to the periphery of the sensing layer.

In an embodiment, the plurality of pressure sensors in the sensing layer are arranged in a first array, the first array including two columns of pressure sensors, each column of pressure sensors being symmetrical and parallel with respect to the user's sagittal axis.

In an embodiment, the plurality of pressure sensors in the sensing layer are further arranged in a second array, wherein the second array is arranged to locate within the pelvis region and includes one or more pairs of pressure sensors, where each of the one or more pairs of pressure sensors are symmetrical with respect to the user's sagittal axis.

In an embodiment, the second array is arranged to locate within the first array.

In an embodiment, the sensing layer is a flexible printed sheet, such that the plurality of sensors and the sensing layer are integrally formed.

In a second aspect, there is provided a system for use with a mobility assistance device, comprising at least one sensing device including a plurality of sensors, the plurality of sensors being in communication with a controller module, wherein the at least one sensing device is arranged to locate between the mobility assistance device and a user and attach to the mobility assistance device so that the at least one sensing device remains in the same position relative to the mobility assistance device, wherein the plurality of sensors collect data that is communicated to the controller module to enable the controller module to determine a state of the user in respect of the mobility assistance device.

In an embodiment, the plurality of sensors includes a plurality of pressure sensors, at least one inertial measurement unit, and at least one strain measurement device.

In an embodiment, the controller module includes a processing module, a memory module, a communication module, an on-board sensor module, a data filter module, a power protection module, and a power access module.

In an embodiment, the on-board sensor module includes a plurality of further sensors selected from the group of; a temperature sensor, a relative humidity sensor, barometric pressure sensor, global positioning system sensor, a magnetometer, a three-axis accelerometer, a three-axis gyroscope, three-axis magnetometer.

In an embodiment, the controller module interrogates at least one of the plurality of sensors and the plurality of further sensors to obtain sensor data, wherein the controller module is configured to undertake data fusion processing on the sensor data.

In an embodiment, the controller module is configured to be part of the sensing device.

In an embodiment, the controller module includes a power source in connection with the power connection module and power access module, wherein the power source includes at least one lithium battery or at least one nickel-metal hydride battery.

In an embodiment, the system further includes an interface module for communicating alerts to the user.

In an embodiment, the system is configured to communicate via the communication module with a mobile device under the control of a user, the mobile device including a user application that collects user data from the user and/or a user's circle of care, wherein the controller module is further configured to undertake the data fusion processing of the sensor data collected by any one of the plurality of sensors, the plurality of further sensors, and the user data, wherein based on the data fusion processing, the controller module classifies user events with respect to the mobility assistance device to determine the state of the user.

In an embodiment, the user application further displays to the user any information relating to the classification of the specific behaviours of the user with respect to the mobility assistance device and the events taken or experienced by the user whilst engaged with mobility and assistance device.

In a third aspect, there is provided a method for determining the state of a user in respect of a mobility assistance device using the system in accordance the second aspect, wherein the method comprising the steps of: communicating data from the plurality of sensors to the controller module, processing the data using the controller module to identify one or more user events, analysing the one or more user events to determine a state of the user, and analysing the state of the user over a plurality of time periods to determine the user's risk metric.

In an embodiment, the method further comprises the step of prompting the user and/or a user's circle of care to alter the user's state by means of an alert if the risk metric reaches a predetermined risk limit by means of a user application.

In an embodiment, the method further comprises the step of alerting the user and/or the user's circle of care that they have reached a goal by means of a user application.

In a fourth aspect, there is provided a method for calibrating a plurality of sensors in a sensing device in communication with a controller module, where the sensing device and the controller module are for use with a mobility assistance device, the method comprising the steps of: undertaking an initial conditioning of each of the plurality of sensors, undertaking an initial calibration to determine the individual performance of each of the plurality of sensors, and undertaking a user calibration to determine the cooperative performance of the plurality of sensors in respect of a user and the mobility assistance device.

In an embodiment, the step of undertaking user calibration further comprises the steps of: undertaking a user conditioning of the plurality of sensors, taking a first reading of the user fully engaged with the mobility assistance device, taking a second reading of the user not engaged with the mobility assistance device, and processing the first and second readings using the controller module and saving the processed first and second readings on the controller module.

In an embodiment, the step of undertaking user calibration further comprises the steps of: taking a third reading of the user partially engaged with the mobility assistance device in a forward direction, taking a fourth reading of the user partially engaged with the mobility assistance device in a right-sided direction, taking a fifth reading of the user partially engaged with the mobility assistance device in a left-sided direction, and processing the third, fourth and fifth readings using the controller module and saving the processed third, fourth and fifth readings on the controller module.

In an embodiment, the method further comprises the step of determining whether the plurality of sensors needs to be re-calibrated.

It is intended that any reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Furthermore, terms such as “front”, “rear”, “top”, “bottom”, “side”, “left’, “right” and the like are only used to describe elements as they relate to one another, but are in no way meant to recite specific orientations of the device, to indicate or imply necessary or required orientations of the device, or to specify how the invention described herein will be used, mounted, displayed, or positioned in use.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting. Where specific integers are mentioned herein, which have known equivalents in the art to which this invention relates; such known equivalents are deemed to be incorporated herein as if individually set forth. As used herein the term ‘(s)’ following a noun means the plural and/or singular form of that noun. Further, as used herein the term ‘and/or’ means ‘and’ or ‘or’, or where the context allows both. The invention consists in the foregoing and also envisages constructions of which the following gives examples only.

Throughout this specification and the claims that follow, unless the context requires otherwise, the word “comprise”, and variations such as “comprises” or “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as, an acknowledgement or admission or any form of suggestion that prior publication (or information derived from it) or known matter forms part of the common general knowledge in the field of endeavour to which this specification relates.

### BRIEF DESCRIPTION OF FIGURES

The present invention is described by way of non-limiting examples within the following description and figures.

FIG. 1A illustrates a partial cross sectional view of a device in accordance with an embodiment of the present invention.

FIG. 1B illustrates a top-down exploded perspective view of a device in accordance with an embodiment of the present invention.

FIG. 1C illustrates a bottom-up exploded perspective view of a device in accordance with an embodiment of the present invention.

FIGS. 1D and 1E respectively illustrate a detailed exploded perspective view and a detailed assembled perspective view of Area A as indicated in FIG. 1B in accordance with an embodiment of the present invention.

FIGS. 2A to 2L illustrate various views of a device in accordance with an embodiment of the present invention.

FIGS. 3A to 3D illustrate plan views of a device in accordance with an embodiment of the present invention.

FIG. 3E illustrates a side view of an example of shear forces experienced by a device in accordance with an embodiment of the present invention.

FIG. 4 illustrates a top view of a system in accordance with an embodiment of the present invention.

FIGS. 5A to 5D respectively illustrate perspective, front, top and side views of a system in accordance with an embodiment of the present invention.

FIG. 6 illustrates a side perspective view of a system in accordance with an embodiment of the present invention.

FIGS. 7A and 7B illustrate a network diagram illustrating an embodiment of the present invention.

FIGS. 8A to 8E illustrates user interfaces in accordance with an embodiment of the present invention.

FIG. 9 illustrates an embodiment of the present invention.

FIG. 10A illustrates an example calibration testing apparatus in accordance with an embodiment of the present invention.

FIGS. 10B and 10C illustrate examples of user interfaces in accordance with an embodiment of the present invention.

FIGS. 11A to 11J illustrates an example user interface in accordance with an embodiment of the present invention.

### DETAILED DESCRIPTION

In broad terms, the present invention provides a device, system and method for determining the state of a user. Within the broader inventive concept, various embodiments of the device are described and defined in further detail below. Further, within the description and the figures, reference to like numbers denotes reference to like features.

Within the context of the specification, the terms used are understood to hold their normal meaning within the art. In particular, the term “attached” may be taken to mean to fasten, join, or connect something in either a permanent or temporary manner. It is understood that when so attached, the relative positions of the objects attached to one another remains the same. The terms “permanent” refers to a lasting form or means of attachment and “temporary” refers to a non-lasting form or means of attachment.

Further, the use of words such as “transmits”, “transfers”, and “communicates” are used interchangeably in referring to the transference of data between devices, systems, network nodes or other such aspects. For example, these terms may be used to refer to the transference of digital data over a computer network, telecommunications network, data communications network, Local Area Network (LAN), Wide Area Network (WAN), wireless network, Ethernet, the Internet and developments thereof, transient or temporary networks, combinations of the above or any other type of network providing for communication between computerised, electronic or digital devices. More than one distinct network can be provided, for example a private and a public network. A network as referenced in this specification should be taken to include any type of terminal or other similar type of electronic device, or part thereof, which is rendered such that it is capable of communicating with at least one other terminal.

Further, the terms “force” and “pressure” are known to be related terms as forces generated by a person's movement or mass create certain pressures on the body relative to a structure or surface. As such, these terms may be used interchangeably.

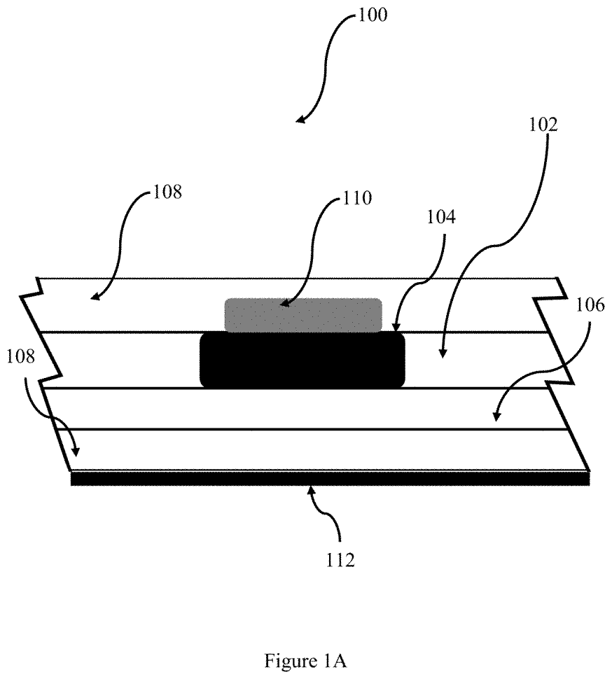

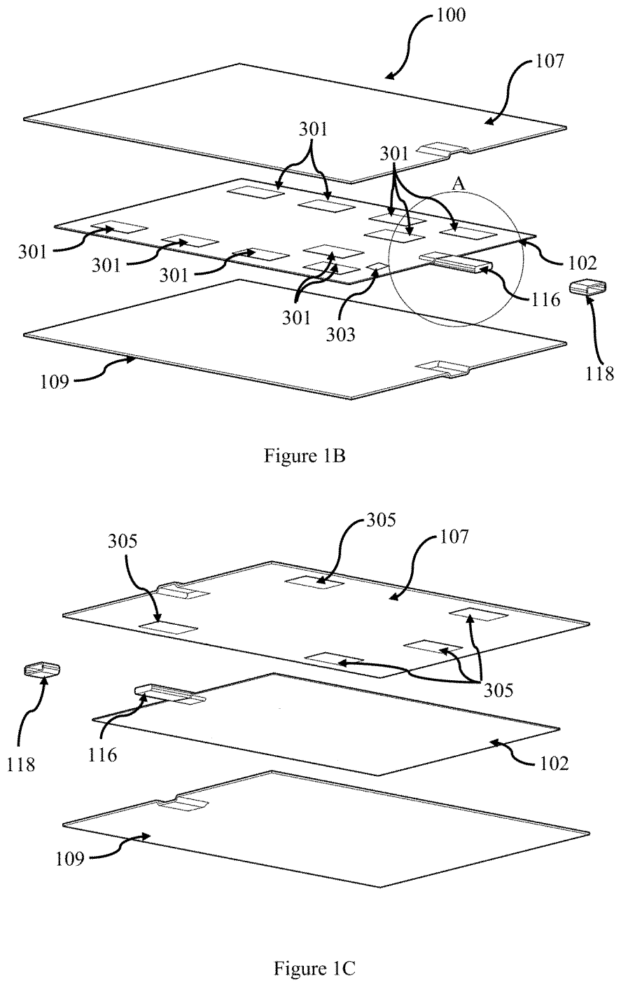

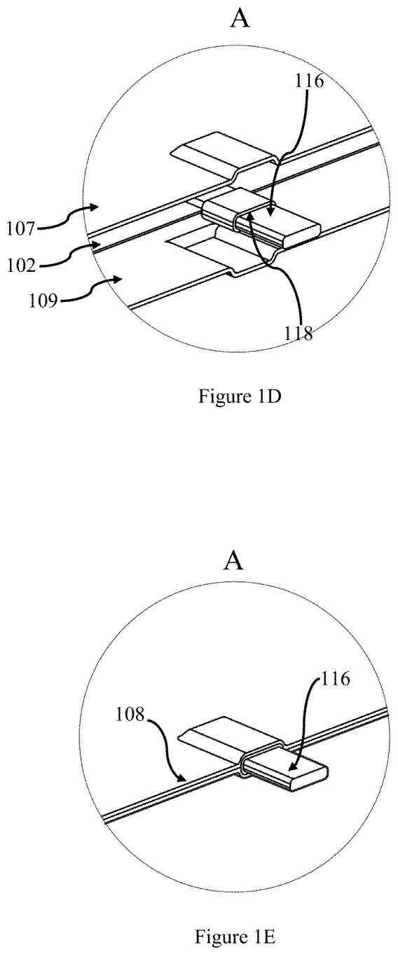

Referring generally to FIGS. 1 to 3E, there is provided an embodiment of the sensing device 100 for use with the mobility assistance device. The mobility assistance device may be a wheelchair, such as a power wheelchair, manual wheelchair, foldable wheelchair that may include removable cushions. Alternatively, the mobility assistance device may be a motorised scooter, knee walker, or other such device that supports the weight of a user whilst enabling the user to support and move themselves using the mobility assistance device. Additionally, the sensing device 100 may be secured to any surface or plane that supports the body to determine and communicate the state of the user. Further, the sensing device 100 may also enable a means to measure the balance of a user or act as a balance or fall detection system.

The sensing device 100 may be comprised of a number of layers, including a sensing layer 102, where the sensing layer 102 includes a plurality of sensors 104 being arranged along at least one plane. The sensing device 100 may further include an outer a top outer layer 107 and a bottom outer layer 109, where the top layer 107 and bottom layer 109 are sealingly arranged to enclose the sensing layer 102. When so arranged, the top layer 107 and bottom layer 109 form an outer layer 108. The sensing device 100 may be arranged to locate between the user and the mobility assistance device, and is configured to attach to the mobility assistance device so that the sensing device 100 remains in the same position relative to the mobility assistance device.

Referring to FIGS. 1A to 1E, an embodiment is provided of the sensing device 100. The sensing layer 102 may comprise a plurality of sensors 104 being arranged along at least one plane. The plurality of sensors 104 include any number sensors that may be proximate with or attached to the sensor layer 102 or integrally formed with the sensor layer 102 as a sensor sheet. The plurality of sensors 104 may include a plurality of force sensors 301, at least one inertial measurement unit (IMU) 303 and at least one strain measurement device 305. These sensors and other sensors included in the plurality of sensors 104 are discussed in further detail later in the specification.

In any of the below described embodiments, the arrangement of the plurality of sensors 104 and sensing device 100 as a whole may be arranged and configured to be appropriately sized for the user. In some cases, this may require increasing or decreasing the sensing areas of the plurality of sensors 104 to provide a layout that is proportionate to the size required by the user and to avoid sensors sitting to close or too far apart.

In an embodiment, the plurality of sensors 104 are included in the sensing layer 102 in such a way as to ensure that their positions are fixed with respect to one another. This is to minimise the risk of changes in the relative positions between sensors causing errors in the collected data. The inclusion of the plurality of sensors 104 within the sensing layer 102 may be undertaken in various ways. Further, the sensing layer 102 may be made from a variety of materials at a variety of thicknesses in order to accommodate the plurality of sensors 104.

For example, the plurality of sensors 104 may be arranged in the sensing layer 102 by means of a material. The material may be a fabric material, for example fusible cloth. The plurality of sensors 104 may be integrated into the fabric material or attached to a surface of the fabric material by means of adhesive, stitching or another suitable method of arranging the plurality of sensors 104 such that they are joined with the fabric in a way that prevents them from moving.

Further, the sensing device 100 may also include a plurality of conductive elements (not shown). The plurality of conductive elements are configured to connect the plurality of sensors 104 and other electronic components together to enable the communication of electrical signals and transmission of power. That is, the conductive elements may include wires, clips, lead solder and the like. In an embodiment including the fabric material as described above, the conductive elements may also be integrated with, or attached to, the fabric material. The wires may terminate at a flexible and durable cable with a secure connection, such as a strain relief, to reduce the stress to the electrical system and connections. For example, the durable cable may be a ribbon cable. The durable cable may connect to a controller module 500, and in doing so, enables communication of the sensing device 100 with the controller module 500. As such, the durable cable supplies the plurality of sensors 104 with power from the controller module 500 and transmits sensors signals to the controller module 500. The controller module 500 is described in further detail later in the specification.

In other embodiments, alternative sensing layer 102 arrangements and materials may be provided. For example, the sensing layer 102 may be formed by encapsulating the plurality of sensors 104 in a material such as silicone, rubber, plastic or a polymeric material. This may be manufactured by injection moulding, thermoforming or other such methods of manufacture. That is, the plurality of sensors 104 and their conductive elements may be integrally formed with the sensing layer 102 to form a flexible electronic circuit. In an embodiment, further electrical components may be mounted on the flexible circuit, where the circuit may include polyimide or transparent conductive polyester film. In an embodiment where the plurality of sensors 104 are formed with the sensing layer 102 in a single flexible sheet, the sheet may include slots, voids or cuts formed in the sensing layer 102 to improve the flexibility of the sensing layer 102 to reduce wear and tear.

In a further embodiment, a screen-printing method may be used to produce the sensing layer 102. The sensing layer 102 may be made from a polymeric substrate, which is formed to contain the plurality of sensors 104, conductive elements and any other electrical components. The conductive elements may be in the form of screen-printed conductive traces and may terminate at the flexible and durable cable, for example a ribbon cable. The durable cable may also include a strain relief device to reduce the stress to the electrical system and connections. The durable cable may connect to a controller module 500, and in doing so, enables communication of the sensing device 100 with the controller module 500. As such, the durable cable supplies the plurality of sensors 104 with power from the controller module 500 and transmits sensors signals to the controller module 500. In an embodiment, the sensing layer 102 may be die cut to achieve the desired flexibility.

Referring to FIGS. 1B to 1D, an example is provided where the sensing layer 102, plurality of sensors 104 and the conductive elements between them are formed into a single flexible sheet 114, such that the plurality of sensors 104 and the sensing layer 102 are integrally formed The flexible sheet 114 may include the flexible and durable cable 116 and a reinforcing collar 118. The reinforcing collar 118 may be arranged to receive the durable cable 116 and locate at the periphery between the top outer layer 107 and bottom outer layer 109. Referring to FIG. 1C, the top outer layer 107 and bottom outer layer 109 may be fused, welded, attached by an adhesive or otherwise bonded together to form and outer layer 108. That is, the top outer layer 107 and bottom outer layer 109 may be sealingly arranged to enclose the sensing layer 102, in turn forming the sensing device 100. The term “sealingly” is taken to mean that the sensing layer 102 is sealed within the outer layer 180, which may be sealed to conventional airtight or watertight measures. The outer layer 108 may be arranged to more effectively convey the pressure provided by the user to the plurality of sensors 104. As such, the outer layer 108 may improve the accuracy of the data collected by the plurality of sensors 104.

The sensing device 100 is likely to be subjected to a significant amount of force that may be sustained for long periods of time or be highly repetitious. Such forces and their application are likely to negatively affect the overall life of the plurality of sensors 104 and increase the risk that the plurality of sensors 104 may become damaged. As such, the sensing device 100 may include a base layer 106 to support the plurality of sensors 104 and reduce the likelihood of damage to the plurality of sensors 104 over time.

Referring again to FIG. 1A, an embodiment is provided where the base layer 106 may be applied underneath each of the plurality of sensor's 104 sensing area. Alternatively, the base layer may be applied to the entire sensing layer 102. The base layer 106 may be made from a material that is relatively more rigid or stiff compared to that of the sensor layer 102 material. The base layer 106 may be formed from materials that are thin and durable and that are compliant with sensor flexure requirements (i.e. do not impede the operation of the plurality of sensors). For example, the base layer 106 may be formed from materials such as but not limited to metals, polymers, or composites. Further, the base layer 106 may aid in providing a flat surface to support the sensors in use and may aid in reducing any shape anomalies present on the mobility assistance device 112.

In an embodiment, the outer layer 108 arranged between the sensing layer 102 and the user of the mobility assistance device 112 may also include one or more pads 110. The one or more pads are configured to align above each of the plurality of sensors 104. The pads 110 may include a plurality of compliant pieces of materials that are placed over the sensing surface that are less conformable than the surrounding material of the outer layer 108. Such an arrangement enables each pad 110 to concentrate any force directly onto each of the pluralities sensor's 104 sensing area, even if such force is small.

The one or more pads 110 may be made from rubber or similar materials with a hardness around or above a Shore Durometer of 30 and scale of A. In an embodiment, the one or more pads 110 may be adhered to the outer layer 108 by means of an adhesive or similar means. In a further embodiment, part of the outer layer 108 where the pads 110 are located is removed to create a cavity capable of receiving the pads 110 so that the outer layer 108 remains uniform and smooth. In such an arrangement, the pads 110 may be retained within the cavities by means of adhesive, a tight fit, or other mechanical means. In an alternate embodiment, the outer layer 108 is formed from a single integral piece of material where the pads 110 are comprised of raised areas that are arranged on the outer layer 108 on top of where the plurality of sensors 104 are to be located. In any of the above embodiment, the surrounding area around the pads 110 and the plurality of sensors 104 may be filled with materials such as felt or silicone to remove any gaps or spaces created by the one or more pucks 110.

The outer layer 108 may provide a thin, durable and waterproof layer to help protect the plurality of sensors 104. As the sensing device 100 may be used in cases where the wheelchair commonly becomes wet or soiled, an embodiment is provided where the outer layer 108 may be comprised of a waterproof material and one that is easily cleaned. Further, the outer layer 108 may also be machine washable. For example, the outer layer 108 may be made from silicone, Polyurethane Laminate (PUL), or Polyvinyl chloride, or other such materials. Alternatively, a combination of such materials may be used to form the outer layer 108. In a further embodiment, the entire sensing device 100 may be assembled using a series of moulds and pours such that all the layers of the sensing device 100 are integrally formed with one another.

It would be understood by the person skilled in the art that further types and arrangement of layers is within the scope of the invention as described and defined in the claims. For example, the sensing device 100 may include fabric or felt material or other similar material to fill in the gaps between layers in order to ensure that the sensing device forces are distributed uniformly across the pad. Further, an additional and separate base layer (not shown) may be provided, which is arranged to provide a smooth base for the sensing device. The additional and separate base layer may be arranged outside any cover or cushioning that may be provided to the sensing device 100. Other such layers or arrangements are provided in order to improve sensor readings, durability and user comfort in respect of the sensing device 100.

As described above, the sensing device 100 may be attached to the mobility assistance device. This may be facilitated by means of one or more mechanical devices. The mechanical devices may permanently attach the sensing device to the mobility assistance device. Alternatively, the mechanical devices may temporarily attach the sensing device to the mobility assistance device, but do so in a manner than ensures that, whilst so arranged, the sensing device 100 remains in the same position relative to the mobility assistance device.

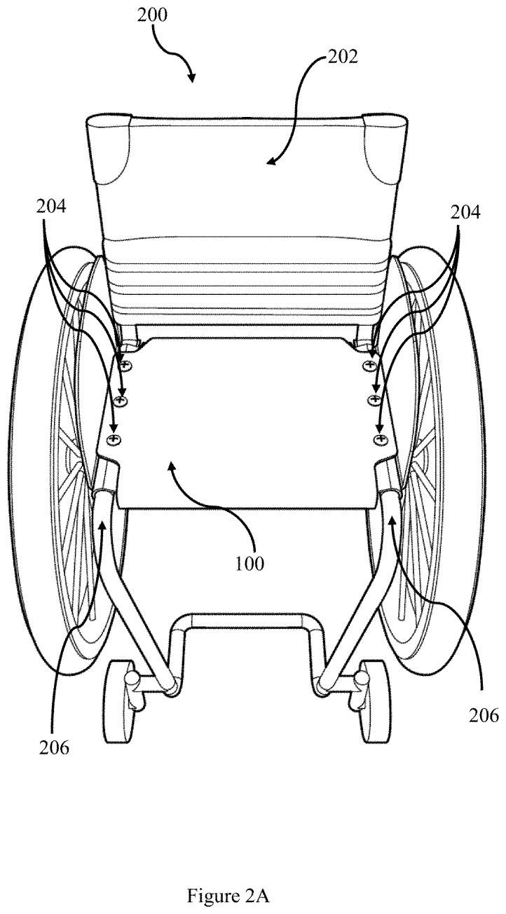

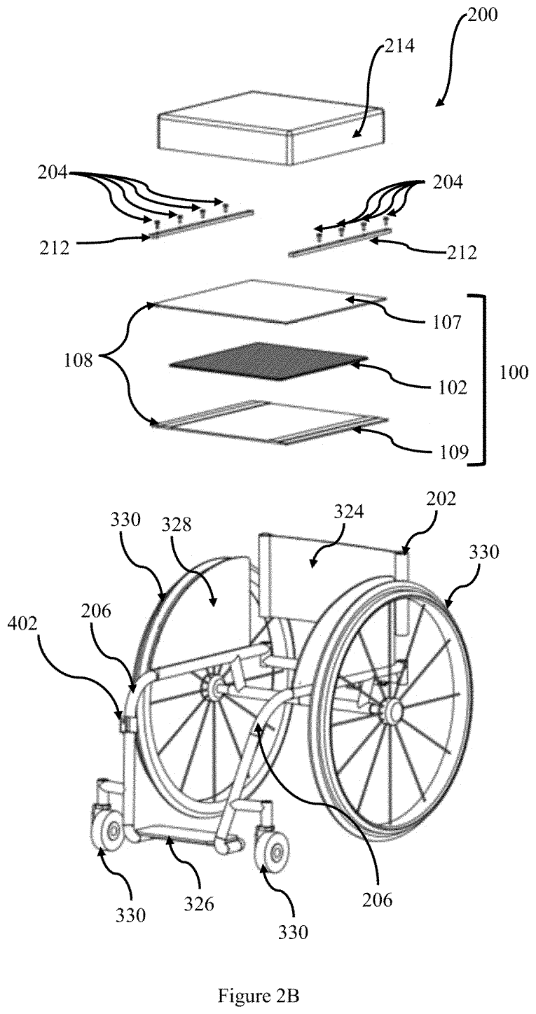

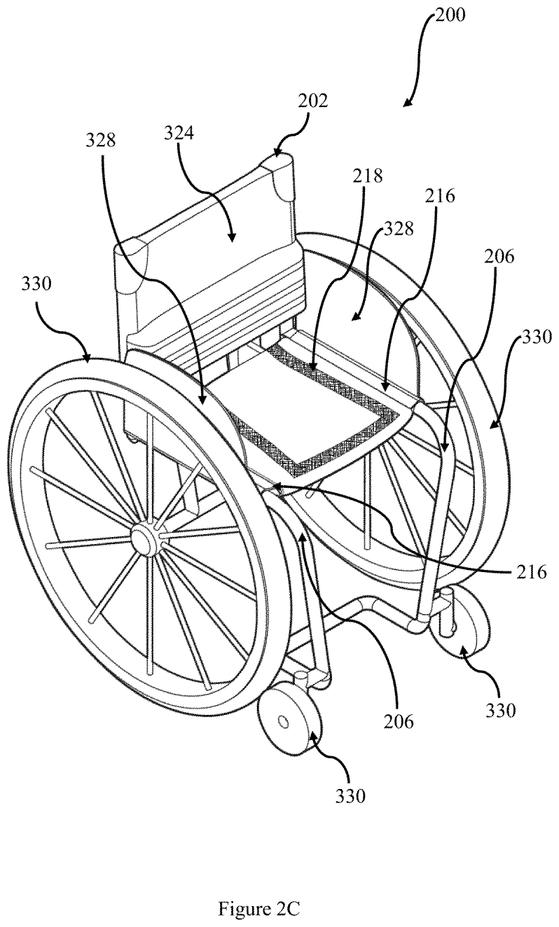

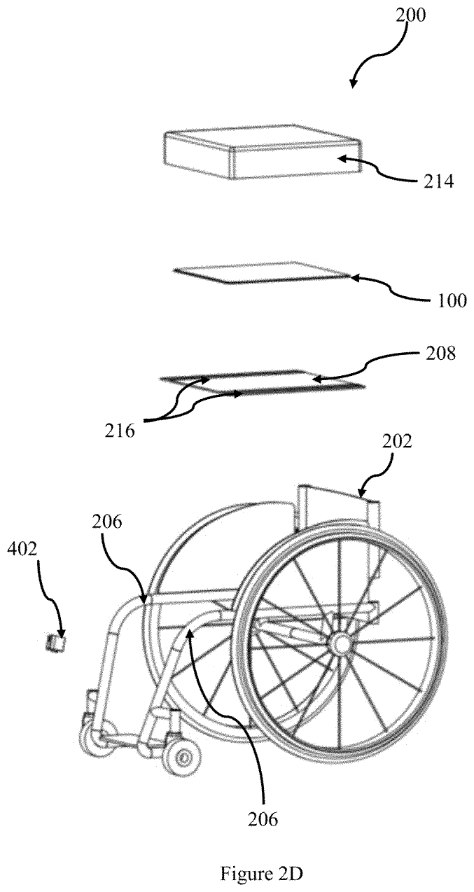

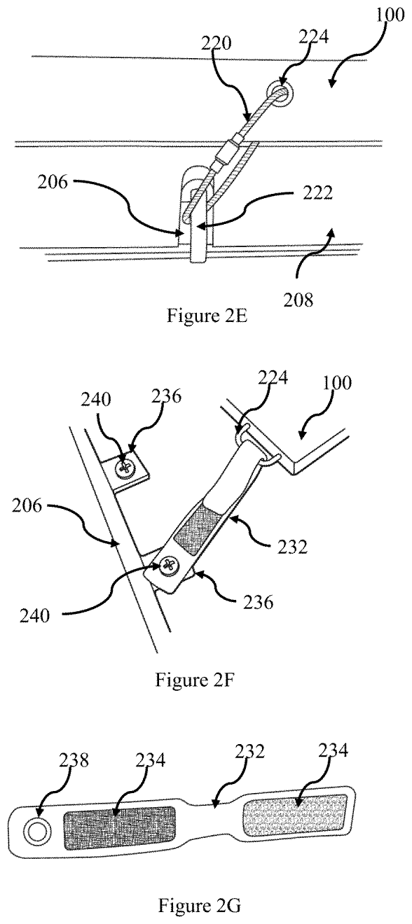

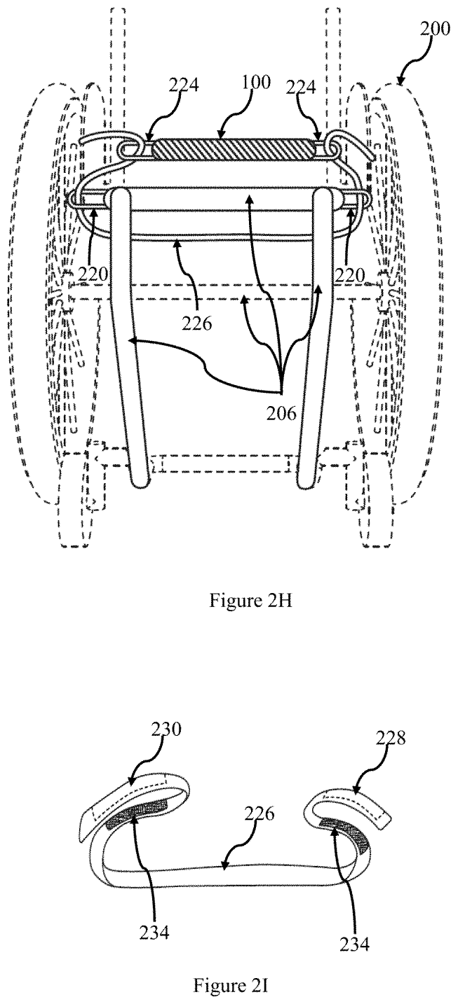

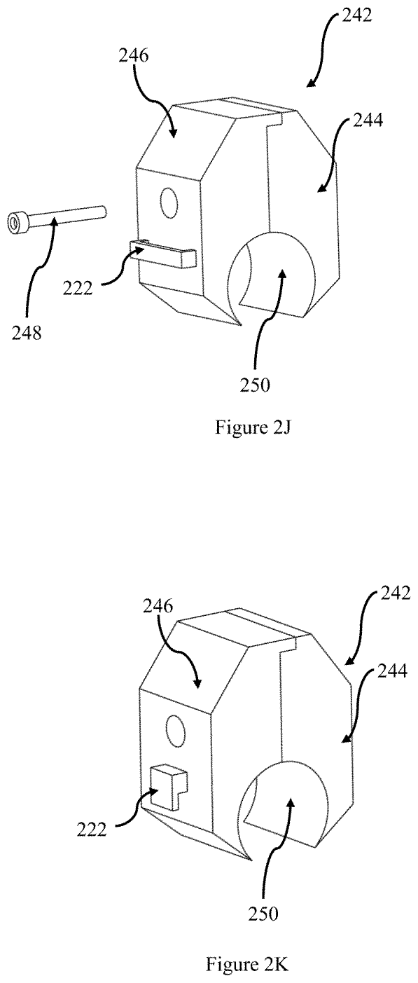

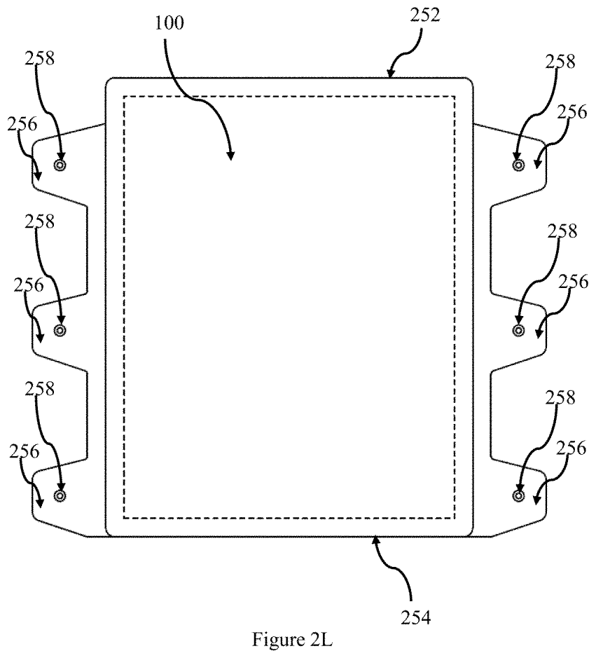

Referring to FIGS. 2A and 2B, an example arrangement 200 is provided wherein the sensing device 100 is attached to a mobility assistance device, where the mobility assistance device is a wheelchair 202. The wheelchair 202 includes a seat frame 206 (referred to as “frame 206”) arranged to support a wheelchair seat. The frame 206 may include multiple struts that connect together to bear the weight of the user. In an embodiment, the sensing device 100 may be configured to attach to the frame 206 to replace the wheelchair seat of a manual wheelchair so that the sensing device is integrally formed with the wheelchair. Such an embodiment may be applied as instructed by a user's wheelchair seating prescription provided by a clinician or when replacing the seat of an already purchased wheelchair.

The sensing device 100 may be attached to a mobility assistance device by means of one or more mechanical devices. Such mechanical devices may include screws or bolts 204 passing through a pair of attachment struts 212 that attach the sides of the sensing layer 202 and the frame 206. The screws 204 pass through the sensing device 100 and the frame 206 of the wheelchair 202 to hold them together. The screws 204, in concert with the pair of attachment struts 212, hold the sensing layer 102 in position with respect to the frame 206 and the wheelchair 202. That is, the sensing device 100 is configured to attach to the frame 206 and replace the wheelchair seat.

The sensing device 100 may also be integrated into a power wheelchair such that the sensing layer is integrally formed into, or otherwise attached to the metallic seat and the controller module 500 connected via a cable to the power source of the power wheelchair by USB port or other means (not shown). Additionally, the data output on the state of the user including notifications may be transferred into the heads-up display on a power wheelchair, by the system's 400 application programming interface (API) service. The system 400 is described in further detail below.

In a further embodiment, a cushion 214 may also be provided to align on top of the sensing device 100, where the cushion may be a waterproof or non-waterproof cushion that may be made from fabric, foam or gel.

Alternatively, the sensing device 100 may be configured to sit on top of the wheelchair seat, where the wheelchair seat may be a rigid seat or a flexible sling seat 208 (referred to as flexible sling 208″ and is best shown in FIG. 2C) provided to the wheelchair 202. In such an embodiment, the wheelchair seat is akin the mobility assistance device layer 112 shown in FIG. 1 and may be integrally formed with the frame 206 or be attached to the frame 206 by means of screws 204 in a similar method to described above.

Referring to FIGS. 2C and 2D, another embodiment is provided where the periphery attachment portions 216 of the sling seat 208 may be looped around the frame 206 and attached to another portion of the sling seat 208. The sensing device 100 may be attached to the sling seat 208 by means of another mechanical device, such as a plurality of hook and loop devices (for example. Velcro) provided to the mobility assistance device and the sensing device 100. As shown, one or more sections of Velcro 218 may be removably attached to the sling seat 208 with corresponding sections of Velcro (not shown) attached to an underside of the sensing device 100. The cushion 214 may also be provided to align on top of the sensing device 100 using portions of Velcro or a similar means.

In another embodiment, the mechanical devices that attach the sensing device 100 to the frame 206 may include zip ties, or other such devices alone or in addition to the above described embodiments. For example in reference to FIG. 2E, the sling shown in FIGS. 2C and 2D is provided, where the sensing device 100 is connected to the sling seat 208 and is also directly connected to the frame 206. The sensing device 100 includes an attachment link 220 that is connected at a first end to a frame attachment anchor 222 and connected to a second end to a device attachment anchor 224. In the embodiment shown, the attachment link 220 is a taut loop of metal or plastic cable, the frame attachment anchor 222 is a metal or plastic loop around a portion of the frame 206 and the device attachment anchor 224 is a metal or plastic reinforced aperture 238 formed in the sensing device 100 (otherwise known as an eyelet or grommet). As would be understood by the skilled addressee, one or more of the above arrangements may be used to connect the sensing device to the wheelchair 202.

Referring to FIGS. 2F and 2G, another example is provided of the attachment link 220 where the attachment link 220 is a flexible strap 232 of strong non-stretch material, such as nylon. FIG. 2F provides a bottom view of the arrangement. The flexible strap 232 includes corresponding Velcro portions 234 on each end to enable the flexible strap 232 to form a loop. FIG. 2F shows the attachment of the flexible strap 232 shown in FIG. 2G, where bracket 236 is formed as a part of the frame 206. Bracket 236 is arranged to align with the reinforced aperture 238 and receive a screw 240, or other fixing device. This arrangement connects the flexible strap 232 to the frame 206, where the flexible strap 232 may be looped through device attachment anchor 224 and secured using the Velcro portions 234.

Referring briefly to FIGS. 2J and 2K, the brackets 236 may be added via the clamp 242 shown later at FIGS. 2J and 2K. In such an arrangement, the clamps 242 are attached to the frame 206 with the flexible strap 232 looping through frame attachment anchor 222.

Referring to FIGS. 2H and 21, an alternative embodiment is provided where, the mechanical devices may include one or more harness straps 226. At first end 228, each harness strap 226 is arranged to pass through a first device attachment anchor 224 provided on the right side of the sensing device 100 and attach to another part of the first end 228 of the harness strap 226. The remainder of the harness strap 226 is arranged to pass around the frame 206, pass underneath the sling seat 208, where a second end 230 of the harness strap 226 is arranged to attach to a second device attachment anchor 224 provided on the left side of the sensing device 100 in the same manner as the first end 228. In an alternate embodiment, the frame may include a frame attachment anchor 220 through which the harness strap 226 may pass.

The one or more straps 226 are arranged to connect to itself by means of Velcro portions 234, stitching or adhesive to form a loop that retains each side of the sensing device 100. Alternatively, the one or more straps 226 may be provided with a Velcro portion 234 on the first end 228 that is attachable to a Velcro portion 234 on the second end 230. That is, the one or more straps may fully encircle the sensing device 100 and the wheelchair 202.

For any of the above described embodiments, the one or more straps 226 may be arranged to be arranged to extend from left to right as shown in FIG. 2H, or may be arranged to extend from front to back (not shown). The one or more straps 226 are tautly arranged to ensure that the sensing device 100 remained in a fixed position with respect to the wheelchair 202.

Referring again to FIGS. 2J and 2K, further mechanical devices are disclosed. In one embodiment, the mechanical devices may include a clamp 242 that is comprised of a first clamp portion 244 and a second clamp portion 246. The first clamp portion 244 and the second clamp portion 246 are held together in a clamped arrangement by means of a pin, bolt or screw member 248. The first clamp portion 244 and the second clamp portion 246 may be configured to clamp around the frame 206 of the wheelchair 202. As such, a clamping face 250 may be provided to engage with the frame 206 and hold the clamp 242 in position on the frame 206. The clamp 242 may include a frame attachment anchor 222, that may be provided as a loop arrangement in FIG. 2J or a tongue arrangement in FIG. 2K, where the frame attachment anchor 222 in either arrangement is configured to attach to the device attachment anchor 224 provided to the sensing device 100, attachment link 220, or flexible strap 232.

With reference to the above paragraphs and aforementioned figures, it would be understood by the skilled addressee that a combination of two or more of the above mechanical devices may be used to secure the sensing device 100 to the mobility assistance device.

Referring to FIG. 2L, an embodiment is provided where the sensing device 100 includes a cover 252, wherein the sensing device 100 is received and retained by the cover 252. That is, the cover 252 may be shaped to form a sealable pocket with an open end 254 for receiving the sensing device 100. The cover 252 may include cushioning or protective elements to reduce the overall wear and tear experienced by the sensing device. For example, the cover 252 may be made from a material that is easily cleaned when soiled.

In an embodiment, the cover 252 may be directly be attached to the mobility assistance device. Such an arrangement may be achieved in a variety of ways in order to suit the arrangement and type of mobility assistance device and the requirements of the user. For example, the cover 252 may include tabs 256, each including a reinforced aperture 258. The reinforced apertures 258 are configured to receive screws or bolts that attach the tabs 256 to the frame 206. Thus, the sensing device 100 may be maintained in a fixed position with respect to the mobility assistance device. In an alternate embodiment, the cover 252 may replace the sling seat 208. Alternatively, another example includes the cover 242, which holds the sensing device 100 and other electrical components (such as the controller module 500) securely in place under or within the cover 242. That is, even when the wheelchair 202 is a foldable wheelchair, or when the cushion 214 is removed, the sensing device 100 and the plurality of sensors remain in a fixed location relative to the wheelchair 202.

The attachment of the sensing device 100 to the mobility assistance device is a non-trivial exercise, as the position of the sensing device 100 must remain fixed relative to the mobility assistance device to ensure accuracy the sensor data. As with most wheelchair accessories available on the market, multiple attachment options are required to address the variety of mobility assistance devices available and to ensure the device is fit for purpose for everyday use such as removing the cushion, disassembling of the wheelchair and requirements to remain cleanable if soiled.

In an embodiment, the plurality of sensors 104 may be arranged in a pattern that is symmetrical along a user's sagittal plane or longitudinal plane, which is an anatomical plane located at the centre of the body and divides the body into right and left halves. That is, the plurality of sensors may be arranged on the left in a manner complementary to how the plurality of sensors are arranged on the right. The pattern seeks to ensure that sufficient sensor coverage is provided, that there are not any large areas where sensors are not present and that the sensors are not placed within direct proximity to areas where the sensor device 100 is connected to the mobility assistance device. As such, it would be understood by a person skilled in the art that many different layouts and arrangements of the plurality of sensors 104 in the arrangement are within the scope of the invention as described and defined in the claims.

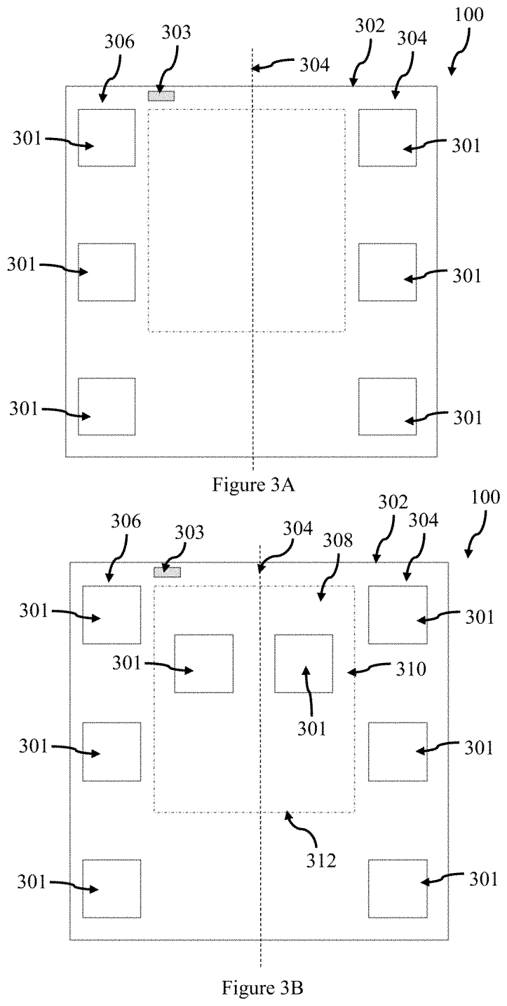

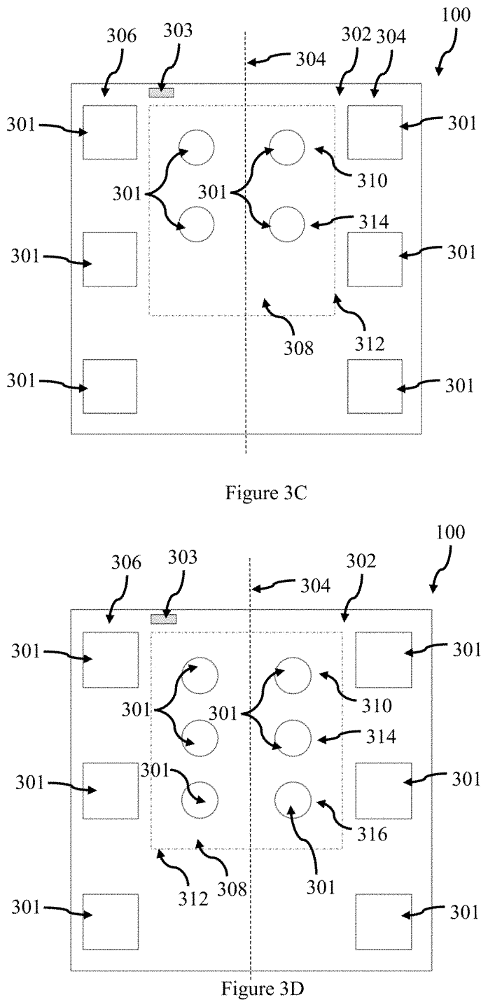

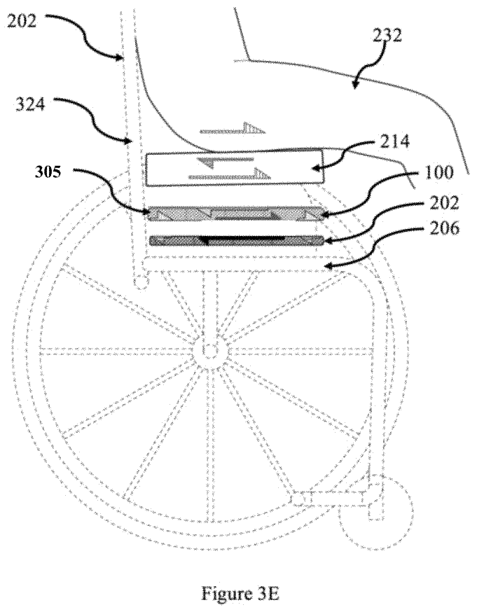

For example, referring to FIGS. 3A to 3D, example sensor layouts are provided for a mobility assistance device being a wheelchair 202, where the layouts includes a plurality of sensors 104. The plurality of sensors 104 may include different types of sensors, which are directed to sensing different types of data. For example, the plurality of sensors 104 may include a plurality of pressure sensors, at least one inertial measurement unit (IMU) and at least one strain measurement device 305, which are discussed in further detail below.

Referring to FIG. 3A, the plurality of pressure sensors 301 in the sensing layer 102 are arranged in a first array 302, the first array 302 including two columns of pressure sensors, a first column 304 and a second column 306. Each column 304, 306 is arranged to be symmetrical and parallel with respect to each other and the axis 304. The user's sagittal plane is indicated by axis 304. The plurality of pressure sensors 301 are understood to be force-sensing sensors that are configured to detect a force applied, where the force applied is a user's body weight.

The use of arrays arranged proximate to anatomical features is advantageous over the use of single sensors placed where the anatomical features are ideally located as it factors in the differences in the body of a user in relation to the user's centred position. This also enables a greater variety in the user body shapes and sizes that are accommodated by the sensing device 100. Further, the location of each of the plurality of sensors 104 in respect to one another is known and precise so that when the information from the plurality of sensors 104 is fused together, the resulting fused data is an accurate representative of the data collected by the plurality of sensors 104.

The IMU 301 may be arranged to detect information such as the wheelchair's 202 movement, orientation, stability, the behaviour of the sling or wheelchair seat, and/or other information relating to the user. In one example, the IMU 301 may be located on the same plane as the plurality of pressure sensors 301 or may be located on another plane or layer within the sensing device 100. Further, the IMU 303 may be located proximate to a power or data connection, such as the controller module resulting in the IMU 303 being located on the periphery of the sensing layer 102 or sensing device 100. In such another embodiment, the IMU may be located at a position along the axis 304 and proximate to the rear side of the wheelchair 202. For example, as shown in FIGS. 3A to 3D, the IMU is located proximate to the top second column 306 at the back of the sensing device 100, although it is not limited to this portion.

Further, the plurality of sensors 104 may include one or more strain measurement devices 305. The at least one strain measurement device 305 is directed to measuring shear strain over the surface of the sensing device 100. For example, the strain measurement device 305 may include, but not be limited to, a strain gauge sensor. The strain measurement device 305 may be arranged in a variety ways to detect a variety of forces, particularly in areas of the sensing device 100 where there is resistance to the shear forces of the body's weight on the sensing device.

For example, the at least one strain measurement device 305 may be attached to a sensing layer facing-side of the top outer layer 107 as shown in FIG. 1C. In this example, five strain measurement device 305 are arranged around the periphery of the top outer layer 107. When so arranged, the gauge sensors 305 may be arranged in a different plane to the IMU 303 and the plurality of pressure sensors 301. Alternatively, as shown in FIG. 3E, the at least one strain measurement device 305 may be arranged to connect between the back support 324, or a rear support portion of the frame 206 and the sensing device 100. In such an arrangement, the at least one strain measurement device 305 may be arranged in the same plane as the plurality of force sensors 301 and the at least one IMU 303.

With continued reference to FIG. 3E, a number of arrows are provided that are indicative of the various reactive shear forces that may be experienced by the wheelchair 202, the sensing device 100 and the cushion 214 when a user 322 is using the wheelchair 202 in accordance with the embodiment shown at FIG. 2B. As such, the strain measurement device 305 may be arranged or located to detect any of the forces indicated by the arrows.

Referring to FIG. 3B, an alternate layout is provided where the sensing device 100 includes the first array of sensors 302 as described above and a second array 308. The second array 308 includes one or more pairs of pressure sensors 310 and the second array 308 is arranged to locate within the pelvis region 312. The pelvis region 312 described the general area on the sensing device 100 indicated by the box marked 312, where the box indicates where a user's pelvis will sit on the sensing device 100. The one or more pairs of pressure sensors 310 are arranged to be symmetrical with respect to the user's sagittal axis. That is, each one of the one or more pairs of pressure sensors 310 is evenly distributed on either side of the axis 304.

A pelvis's anatomical features of the seatbones and tailbones that create signature pressures are bounded on either side by the user's thighs. As such, the pelvic region 312 is arranged to locate between the columns of pressure sensors 304 and 306. This means that the second array 308 is arranged to locate within the first array 302.

Referring to FIG. 3C, a further example of the sensing device 100 is provided, wherein the first array 302 is arranged as described above. However, the second array 308 includes a first pair of pressure sensors 310 and a second pair of pressure sensors 314. The first and second pairs of pressure sensors 310, 314 are located within the pelvis region 312. Referring to FIG. 3D, yet another example layout of the sensing device 100 is provided, wherein the first array 302 is arranged as described above. However, the second array 308 includes a first pair of pressure sensors 310, a second pair of pressure sensors 314 and a third pair of pressure sensors 316. The first, second and third pairs of pressure sensors 310, 314, 316 are located within the pelvis region 312.

In a further embodiment, other sensors may also be included in the sensing device 100. For example, such further sensors may include additional pressure sensors, IMUs, strain gauges sensors, or flex sensors, temperature sensors, a magnetometer, relative humidity sensors, barometric pressure sensors, tilt sensors, vibration sensors, Global Positioning System (GPS), 3-axis accelerometers, 3-axis gyroscopes, 3-axis magnetometer, a combination of all three as a 9-axis motion tracking device, also referred to as the IMU, or other similar types of sensors.

The sensor layouts described above at FIGS. 3A to 3D enable the tracking of general movement, pressure and pressure reliefs, arrangement of the pelvis and whether the user is ideally seated in the wheelchair 202. The arrangement of the pelvis may include the positions of neutral pelvis posture, anterior pelvic tilt, posterior pelvic tilt, pelvic obliquity, pelvic rotation or a windswept posture.

In another example, the plurality of sensors may be arranged in a circular shape (not shown). For example, the plurality of sensors may include ten or eight sensors arranged in a circle or a series of concentric circles. In a further example, the plurality of sensors may be arranged in a square shape or in an oval shape. As such, the arrangement of the plurality of sensors may vary depending on the needs of the user and/or the size, shape and function of the mobility assistance device. Further, the plurality of pressure sensors 301 in the FIGS. 3A and 3B are represented by square shaped sensors and the plurality of pressure sensors 301 in the second array 308 in FIGS. 3C and 3D are represented by circular shaped sensors. However, it would be understood that the sensors may take other shapes, such as rectangles, ellipses or other complex shapes as required.

The above-described layouts of the plurality of sensors are optimised to determine the state of the user when using the mobility assistance device. Use of an optimised layout and providing an arrangement of the sensing device that is in fixed location with respect to the wheelchair increases the accuracy of the data collected from the plurality of sensors. This data may then be then collected by the system 400, where the system 400 uses the data to create a model of the user's seating behaviours to determine whether their behaviours creates a health risk and communicate such risk to the user and/or their permissioned circle of care.

A wheelchair user (referred to as “the user”) may be supported in their activities of daily living, health management and functional recovery by a “circle of care” which can include primary carers, support workers and clinicians. The timely communication of the state of the user including the risk metric of each user is critical to managing the user's health and quality of life. Further, the inclusion of the circle of care in such communications may enable them to inform the classification of the state of the user and user risk in the user application by entering health information, observations and care plan thresholds.

In an embodiment, the pressure sensors in the above described embodiments may include force-sensing resistors and/or force-sensing capacitors. In other words, the pressure sensors may include of a conductive polymer material connected in a circuit, wherein the electrical resistance/capacitance of the polymer material varies according to the application of force to the surface of the polymer material, thus allowing for the measurement of the force applied. In an example, each of the plurality of sensors may be a force-sensing resistor or a force-sensing capacitor. Alternatively, the pressure sensors may include any one or more other sensors types that are configured to measure force, such as magnetic, inductive, capacitive, and optical sensors.

Further, such sensors as those listed above, may also be included in the controller module 500 or attached to the mobility assistance device itself. For example, one or more IMUs may be included in the controller module 500 or a separate wearable device (not shown). In another example, a tilt meter, vibration sensors and/or a GPS sensor may be included in the wheelchair 202 or the controller module 500. That is, a further plurality of sensors 738 may be provided, where the further plurality of sensors 738 includes any of the above sensors and are included in the controller module 500 or provided to the mobility assistance device.

In addition to sensors, other electronic components may also be integrated into the sensing device 100. For example, the sensing device 100 may include a radio-frequency identification (RFID) device that may be used to identify the sensing device 100 and its specifications (not shown). The RFID device may be configured to contain information relating to the particular sensor positions, sensing device size, arrangements, channels, force ratings and sensitivity of the sensing device 100 and have this information able to be read by and RFID reader and displayed to a user. For example, the sensing device 100 may include a hierarchy of resistors values that can be measured by the controller module 500 to identify different standard sensing device 100 types. In an embodiment the sensing device 100 details may be tracked using a unique digital serial number component may be integrated in the sensing device 100 to identify the individual sensing device 100, for example using an embedded RFID device. The unique serial number identified by such techniques, may be automatically referenced to the sensing device information held in a database, which may be queried to obtain all information about the sensing device 100 including unique calibration information. Further, the sensing device 100 may include different ports (i.e. charging or data transference) or visual indicators for indicating the state of operation of the sensing device (i.e. Light Emitting Diode (LED) devices) to improve the accessibility and usability.

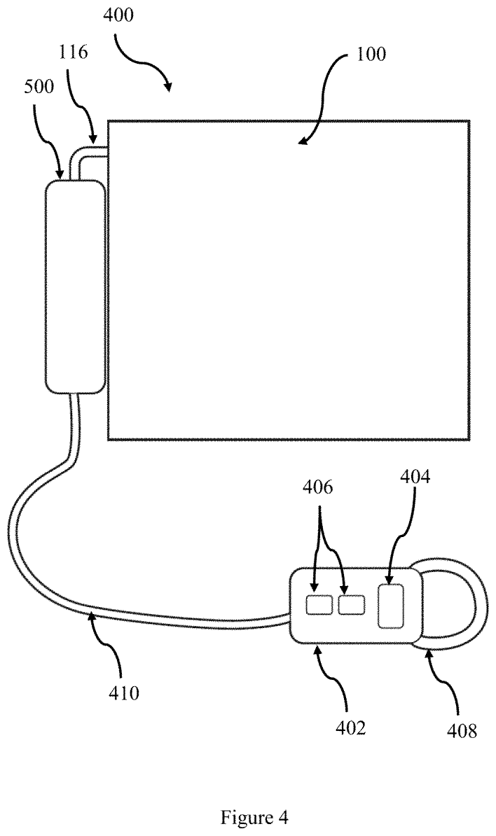

Referring now to FIG. 4, an embodiment is provided of a system 400 for use with a mobility assistance device. The apparatus 400 may comprise at least one sensing device 100 including the plurality of sensors 104 in communication with a controller module 500. The plurality of sensors 104 of the sensing device 100 may be in communication with the controller module 500 by means of a wired connection, such as the durable cable 116. Alternatively, the plurality of sensors 104 of the sensing device 100 may be in wireless communication with the controller module 500.

The at least one sensing device 100 may be arranged to locate between the mobility assistance device and a user such that the plurality of sensors 104 collect data that is communicated to the controller module 500. The controller module 500 uses that data to determine a state of the user in respect of the mobility assistance device. The phrase “state of the user” is used to refer to the seating behaviour of the user in respect to the mobility assistance device or events experienced by the user. Such behaviours or events may include the use of the mobility assistance device, seated location, body position and/or movements, the user performing a pressure/pressure relief, current activity/movement, or health related events such as a spasm or fall. Other examples of user behaviours or events may be described in further detail in the specification below.

The system 400 may include at least one sensing device 100 as described above. Where the mobility assistance device is the wheelchair 202, the sensing device 100 may be arranged to be included in the wheelchair seat or be integrally formed into the wheelchair 202 so as to replace the wheelchair seat as described above. Additionally, the at least one sensing device 100 may be integrated into the wheelchair 202 at other locations, such as in the back support 324, footrest 326, sideguards 328, lateral or head supports (not shown), in the cushion 214, in a cushion cover (not shown) provided to the cushion 214 or in the wheels 330 of the wheelchair 202, where such features are shown best in FIGS. 2B and 2C. Further, where a plurality of sensing device 100 are used, the plurality of sensing devices 100 may be networked together in the system 400 to determine and communicate a fuller understanding of the state of the user and the user's risk metric.

In an embodiment, the system 400 includes a controller module 500 where the sensing device 100 is configured to be in communication with the controller module 500. The controller module 500 controls the hardware of the sensing device 100, particularly in relation to collecting signals, processing, receiving and transmitting information. In order to undertake these processes, the controller module 500 includes a number of sub-components or modules. The modules may include one or more of the following:
a. A processing module including a microcontroller, which is a small computer on a single integrated circuit. The processing module runs the firmware, undertakes at least some of the required processing, and computational requests on the controller module 500 itself. The processing module may contain one or more Central Processing Units (CPUs), memory, programmable input/output peripherals, and Random Access Memory.
b. A memory module, including expanded flash memory, which may be used to store and retrieve data, particularly in instances where data needs to be stored for later transference.
c. A communication module including a Bluetooth low energy module, where Bluetooth is a wireless technology standard for exchanging data over short distances using short-wavelength UHF radio waves for mobile devices and building personal area networks used for relaying real-time data such as alerts or calibration data directly to or from a user's mobile device. The communication module may also include a Cellular and/or WiFi module, which enables WiFi and Cellular connectivity to provide high throughput data transfer channel for sending information such a raw data or receiving firmware updates.
d. An on-board sensor module including the above mentioned further plurality of sensors 738, which may include a temperature and Relative Humidity (RH) Sensor that collects data on the environmental or ambient conditions related to the user. The RH sensor may include a hygrometer to measure the humidity and water vapour. The RH is the ratio of the partial pressure of water vapour to the equilibrium vapour pressure of water at a given temperature. Such data may be used to determine the likelihood of developing certain skin conditions and pressure injury risk. The sensor module may also include a 3-axis accelerometer, 3-axis gyroscope, and 3-axis magnetometer, or a combination of all three as a 9-axis motion tracking device (also referred to as the IMU), which collects data on the movement of the mobility assistance device under the control of the user.
e. A data filter module, which may include an analog pressure sensor filter, which is a specialized circuit for calibrating pressure sensors and filtering raw data so only significant changes in force are passed to the processing module for processing.
f. A power protection module, which may include specialized circuit for protecting a power source in connection with the controller module 500 and intelligently measuring its remaining voltage taking into consideration specific discharge curve.
g. A power access module, which may include a power supply port and indicator, which allows the user to place the charging port and power indicator in a place of their choosing for easy access. The power supply port and indicator and controller module 500 may be connected by a flexible wire or cable, wherein the battery port and indicator are connected to the power source, such as a battery.

One or more of the above components may be integrated into a circuit board, wherein the circuit board includes connections to the secure connection provided to the substrate and the plurality of sensors, power source and include a serial communication port, such as but not limited to a Universal Serial Bus (USB) port suitable for receiving a serial communication link to enable the uploading of software, updates and undertaking testing and troubleshooting.

Referring to FIG. 4, an embodiment is provided where the controller module 500 may be in connection with an accessible interface module 402. The interface module 402 may include a lengthy and flexible interface cable 410 to the controller module 500, such that the interface module 402 may be arranged to be accessible anywhere on the mobility assistance device for improved usability and versatility of installation across the variety of all mobility assistance devices.

Alternatively, in reference to FIGS. 2B and 2D, an alternative embodiment is provided where the interface module 402 is a wireless device that is arranged in wireless connection with the controller module 500. The wireless interface module 402 may communicate with the control module 500 via Bluetooth, Bluetooth Low Energy (BLE) or another short-range communication means.

The interface module 402 may include at least one port 404 capable of receiving a power supply connection for powering the controller module 500 or recharging a rechargeable power source in connection with the controller module 500. The interface module 402 may also include one or more LED device indicators 406 for displaying information relating to the status of the sensing device 100 and/or the controller module 500, such as Bluetooth or WIFI connectivity status or power status. The charging port and display module may also include an attachment bracket 408 to enable attachment to the mobility assistance device. In the embodiment shown, the attachment bracket 408 is looped shaped. However, other shapes may also be used, such as a hook, or sliding arrangement with a cooperative received attached to the wheelchair 202 and/or controller module 500.

In an embodiment, the controller module 500 may be configured to be part of the sensing device 100. Such an arrangement may be provided where the controller module 500 (not including any power sources) may be a further flexible circuit that is integrated within the flexible circuit of the sensing layer 102. Alternatively, the controller module 500 may be configured to be part of the sensing device 100 where the controller module 500 may be configured to be attached to the sensing device 100 and be located within a protected (i.e. cushioned, robust and waterproof) portion of the cover 252.

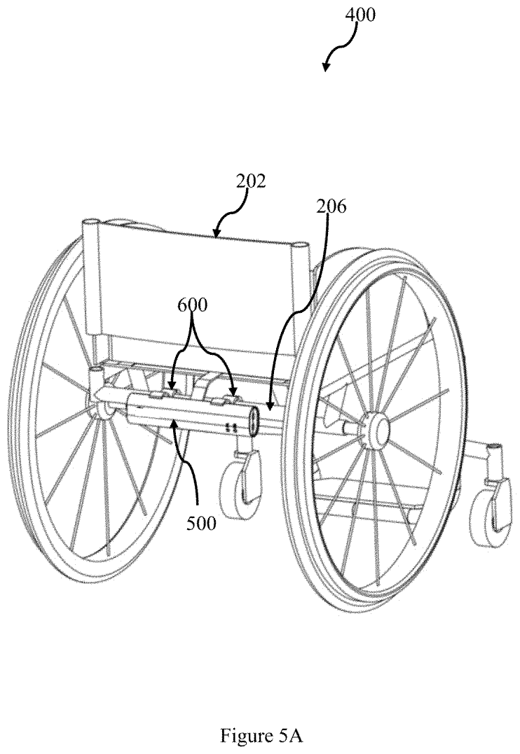

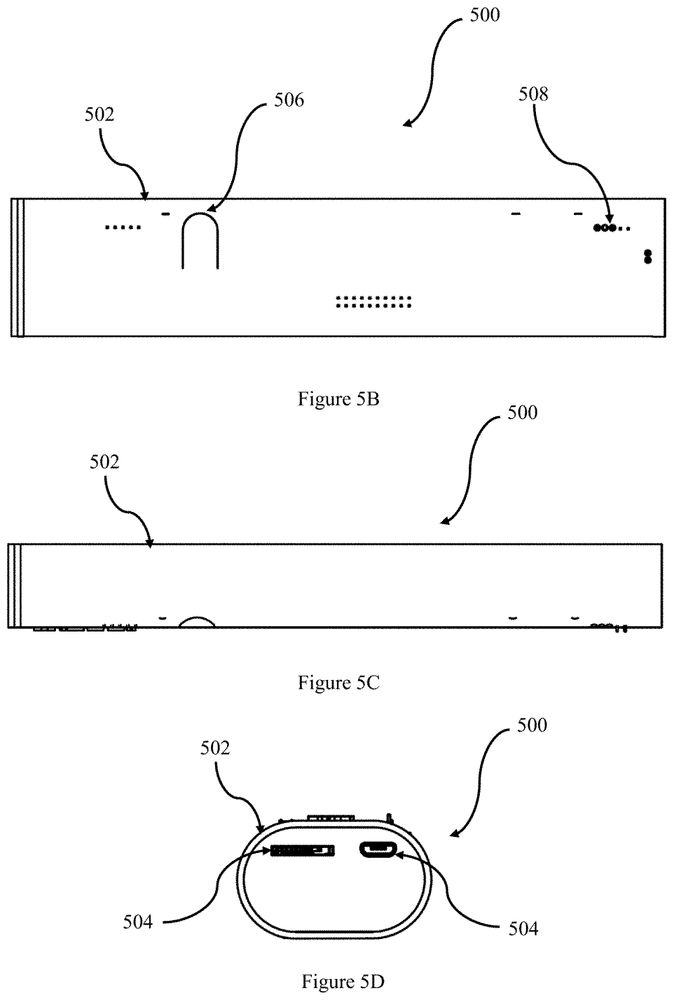

Alternatively, with reference to FIGS. 5A to 5D, the controller module 500 may be separate to the sensing device 100. In such an embodiment, the controller module 500 may include a protective casing 502. The protective casing 502 may be attached to the mobility assistance device in any location that is accessible by the user and will not impede the user's use of the mobility assistance device. For example, where the mobility assistance device is a wheelchair 202, the protective casing 502 may be provided on the back or underside of the wheelchair 202. The protective casing 502 may be formed from a waterproof or weatherproof material that houses and protects the controller module 500, such as plastic or metal. The protective casing 502 may also include cushioning and/or a hard outer shell to protect the power source from physical damage.

The protective casing 502 may also include other features, such as but not limited to, a passive heat exchange, such as a heat sink, or ventilation openings (not shown) to dissipate unwanted heat from electrical components. The protective casing 502 may include one or more data or power ports 504 that are in connection with the communication module of the controller module 500, which enable direct interfacing with the controller module 500 via a wired connection. Further, the protective casing 502 may include a power indicator 506 and processing indicators 508, which may include LED devices that may be programmed to indicate to a user different when the sensing device 100 is in a certain state.

For example, the power indicator 506 may indicate to a user using specific colours or patterns of flashing where the sensing device 100 and/or controller module 500 is on and operating, the sensing device 100 and/or controller module 500 being in need of charging and the sensing device 100 and/or controller module 500 being charged. Similarly, the processing indicators 406 may indicate to a user using specific colours or patterns of flashing where the sensing device 100 and/or controller module 500 is uploading data to another device on a network or is receiving firmware updates.

Referring to FIG. 6, there is provided an example of a controller attachment bracket 600, which is arranged to connect the protective casing 502 to the wheelchair 206. The controller attachment bracket 600 may include a first clamping portion 602 and a second clamping portion 604, where the clamping portions 602, 604 are configured to at least partially clamp around a vertical or horizontal strut of the frame 206 of a wheelchair 202, for example the frame 206 shown in FIG. 5A. A pin, bolt or screw member 606 is arranged to pass through both clamping portions 602, 604, such that the bracket at 600 and is held securely in place on the frame 206.

The bracket 606 may include attachment arms 608 that are arranged to engage with the protective casing 502 and hold the controller module 500 in a secure position on the wheelchair 202. As shown in FIG. 5A, the controller module 500 may be connected to the back of the wheelchair 202. Alternatively, the controller module 500 may be connected to the sides, front, underside or other part of the wheelchair 202.

In an embodiment, the controller module 500 may include a power source. The power source may also be housed within the protective casing 502. The power source may be in connection with the power protection module and power access module of the controller module 500. The power source may include one or more batteries, which may be rechargeable or single use. Such rechargeable batteries may be but are not limited to, Lithium Polymer batteries or Nickle-Metal Hydride batteries. The one or more batteries may be arranged in parallel or series. The one or more batteries may be arranged on the mobility assistance device. For example, the one or more batteries may be housed away from the user. For example, where the mobility assistance device is a wheelchair, the one or more batteries may be arranged on the back of the wheelchair. Alternatively, the controller module 500 may be charged with a magnetic charge cable (not shown) to ensure that if the mobility device is moved away from the charging port, the cable will easily detach without risk to the user or the sensing device 100.

In an alternate embodiment, the one or more batteries may be housed in an additional protective casing (not shown). The additional protective casing also may include a waterproof or weatherproof coating or sheath, and may include other features, such as but not limited to, a passive heat exchange, such as a heat sink, or ventilation openings to dissipate unwanted heat from the power source. The additional protective casing may also include cushioning and/or a hard outer shell to protect the power source from physical damage. That is, the additional protective casing may be a dedicated power source casing that includes many of the features of the protective casing 502 as described above.

In an embodiment, a method may be provided for determining the state of a user in respect of a mobility assistance device. The method may comprise the steps of: communicating data from the plurality of sensors 104 to the controller module 500, processing the data using the controller module to identify one or more user events, analysing the one or more user events to determine a state of the user, and analysing the state of the user over the plurality of time periods to determine the user's risk level or risk metric. Each of these steps is discussed in further detail in the following paragraphs.

In an embodiment, controller module 500 interrogates the plurality of sensors 104 to obtain one or more data sets. The controller module 500 may also interrogate the further plurality of sensors 738 to obtain the data sets. The data sets are communicated from the plurality of sensors 104 and the further plurality of sensors 738 to the controller module 500 over a wired or wireless connection. For example, the data from the sensing device 100 is communicated over cable 116 between the sensing device 100 and the controller module 500.

In an embodiment, the controller module 500 may undertake pre-processing of the raw sensor data, where the pre-processing may include analog filtering. The aforementioned processing module may include an integrated circuit with an analog to digital converter chip that is used to filter the raw data coming from the plurality of sensors. In an embodiment, the processing module includes a temporal filter for large changes within a very short time frame (less than one second). For example, in cases where a large change occurs over a time period of less than one second, the temporal filter will limit the recording of any significant changes to a frequency of one second or more in order to accumulate more information such that the value representing the greatest change will be recorded within the prescribed range. Further, the one-second filter may record the mean, minimum, maximum and standard deviation within the prescribed range.

The analog filtering process may pass signals through to the processing module, which will otherwise remain asleep or on a low power mode unless there has been a significant change, to reduce the re-recording of non-changing values. For example, in the case the user has left the mobility assistance device or is sitting very still, and readings are remaining a constant value. In an embodiment, an interrupt may also be used to reduce the re-recording of non-changing values. An interrupt is a signal to the processor emitted by hardware or software indicating an event that needs immediate attention.

The application of such filtering helps to capture only the data needed to develop user metrics. The benefit of this is this process significantly reduces the data required to be transferred over Wi-Fi or Bluetooth where the efficiency of such transfer directly enhances the ability of the system to communicate the state of the user in timely manner above all known devices. Based on tests conducted with average users setting a filter to only track changes when they exceed 0.5% of the total signal can reduce data volume by over 50%.

Further, an interrupt may also be used to reduce the re-recording of non-changing values for other sensors, such as but not limited to the accelerometer, temperature, RH, and fuel gauge IC. The use of such filters and interrupts enables the system to minimize the data volume and associated storage and transmission requirements while significantly extending battery life.

In one embodiment, the data is processed by the controller module 500 to determine the state of the user, before being stored for further analysis by the further computing system. For example, the controller module 500 may be programmed to recognize certain events associated with the user's activity. For example, an event may be identified by comparison of sensor data to pre-set thresholds. Alternatively, an event may be identified by more complex machine learning based algorithms, which are trained on past data to accurately detect user events. User events are described in further detail later in the specification. This can eliminate the need for sending raw data altogether in cases such as the IMU which can produce nine readings at 100 Hz or more.

In an embodiment, if the controller module 500 is unable to immediately transfer the data, the data may be temporally stored within the flash memory, wherein the controller module 500 may be configured to store the information related to the controller module 500 identifying an event when a new piece of raw data is sent to processing module either from the sensors or based on an interrupt. Once detected, each user event will be logged on the controller module 500 and stored where it can be accessed by a further computing system for further processing

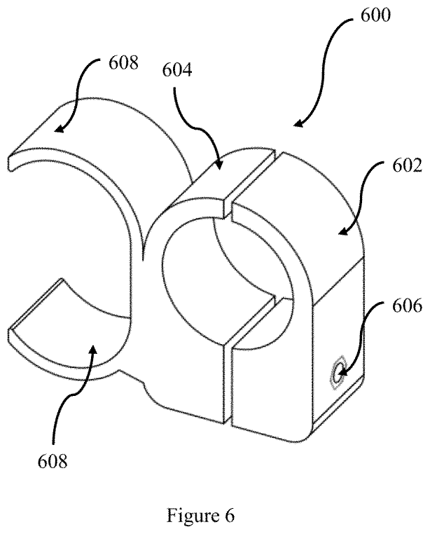

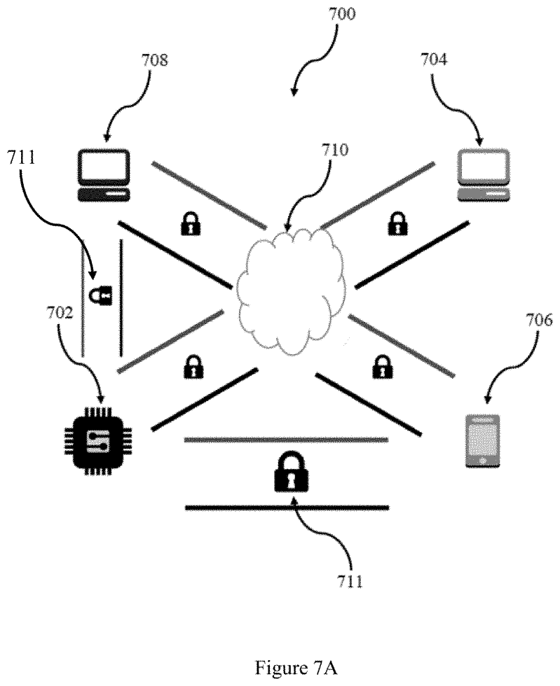

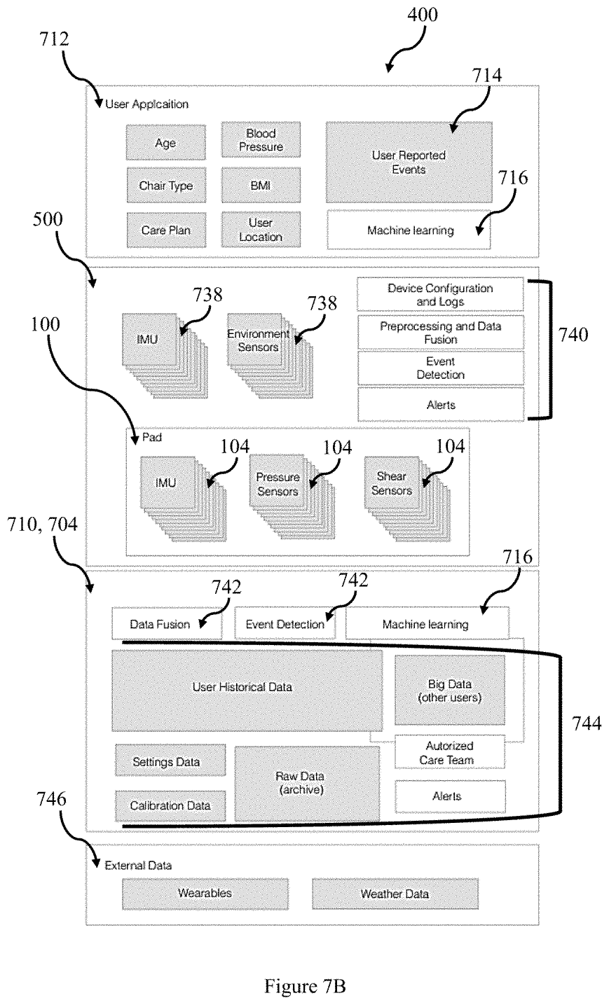

Referring now to FIG. 7A, an embodiment is provided showing a network architecture 700 for carrying out the above-described method using the aforementioned system 400. In an embodiment the method may further comprise the step of storing the data collected and processed by the system 400 on hardware 702 (for example the hardware 702 may include the sensing device 100 and the controller module 500). The stored data may be accessed by and/or transferred to one or more further computing systems over a communication network (for example, via the Internet) for further analysis, processing or presenting to a user.

The one or further computing systems may include the same or different types of further computing systems, which are described in further detail below. In an embodiment, the controller module 500 may be configured to transmit the data to remote cloud service 710. For example, the data from the hardware 702 may be transmitted to remote cloud service 710, which may include secure cloud-based storage or a remote secure server via a wireless or cabled network connection using a secure messaging protocol such as Message Queuing Telemetry Transport (MQTT), which is a publish-subscribe-based messaging protocol. The cloud-based storage or a secure server may be further arranged to enable further processing to determine the state of the user. In a further embodiment, the hardware 702 data is transmitted over Bluetooth to an interim computing system (not shown) before being uploaded to the cloud based storage as needed via the interim computing system's own connection to the cloud-based storage. For example, the controller module 500 transmits the data to the user's computer, where the computer stores the data and later uploads the data to the cloud-based storage.

Alternatively, the one or more further computing system may include a remote terminal 704 such as a generic or specialist computing system that is capable of accessing/retrieving and analysing the data stored in remote cloud service 710. The remote terminal 704 may access the data stored in the remote cloud service 710 via a wireless or cabled network connection, such as the Internet, using a secure messaging protocol such as Hypertext Transfer Protocol Secure (HTTPS). The remote terminal 704 may include a user interface (UI) that is used by either the user or the user's circle of care. The UI may also be arranged to display any results of the further analysis and information relating to the state of the user to the user or the user's clinician. Further, the UI may be arranged to display raw sensor data in a graphical or visual form. This may be enabled by means of a web based API that enables the user and/or the user's circle of care to use a web application, such as a browser application to securely access, in real time, the information relating to the state of the user.

In an embodiment, the further computing system may also include a mobile device 706 such as a tablet or smart phone. The mobile device 506 may access the data stored in remote cloud service 710 via a wireless or cabled network connection using a secure messaging protocol such as MQTT or HTTPS. The mobile device 704 may include a UI that is used by either the user or the user's clinician. The UI may also be arranged to display any results of the further analysis and record information relating to the state of the user to the user and/or the user's circle of care. Further, the UI may be arranged to display raw sensor data in a graphical or visual form. This may be enabled by means of a web based API, which enables the use to use a web application, such as a browser application to securely access the information.

Alternatively, the hardware 702 may be configured to communicate directly with the mobile device 706. For example, this may be enabled by means of a specific mobile user application 712 (shown in FIG. 7B) or “app” installed on the mobile device 706 that is configured to directly display and process real time information on the state of the user from the hardware 702 on the mobile device 706. In an embodiment, the controller module 500 (as part of the hardware 702) may communicate directly with the mobile device 706 over a secure personal wireless network 711, such as a passcode encrypted Bluetooth or BLE network.

The further computing system may also include a moderator device 708 under the operation of an authorised software developer associated with the system 400 that enables the software developer to access and correct any issues that arise with the data. The moderator device 708 may access the data stored in remote cloud service 710 via a wireless or cabled network connection, using a secure messaging protocol such as HTTPS by means of a web based API, which enables the use to use a web application, such as a browser application. Alternatively, the moderator device 708 may communicate directly with the hardware 702 to access the information over a secure personal wireless network 711.

Referring now to FIG. 7B, components of the system 400 are illustrated. In the example provided, the system 400 includes a user application 712 in the form of a mobile user application provided to a user's mobile device 706. The user application 712 may enable the user to set up a user profile and include their own specific characteristics within that profile. This may be guided or completed by a primary carer or clinician on behalf of the user. For example, the user profile may include the user's age, weight, level of injury, date of injury, wheelchair and cushion type and dimensions, care plan, location, body mass index (BMI), blood pressure and other such characteristics.

In an embodiment, the user application 712 is configured to enable the user to enter data events associated with their activities of daily living by means of a user reported events module 714. That is, the step of collecting data from the plurality of sensors may further include collecting data entered by the user and/or the user's circle of care on the user's state.

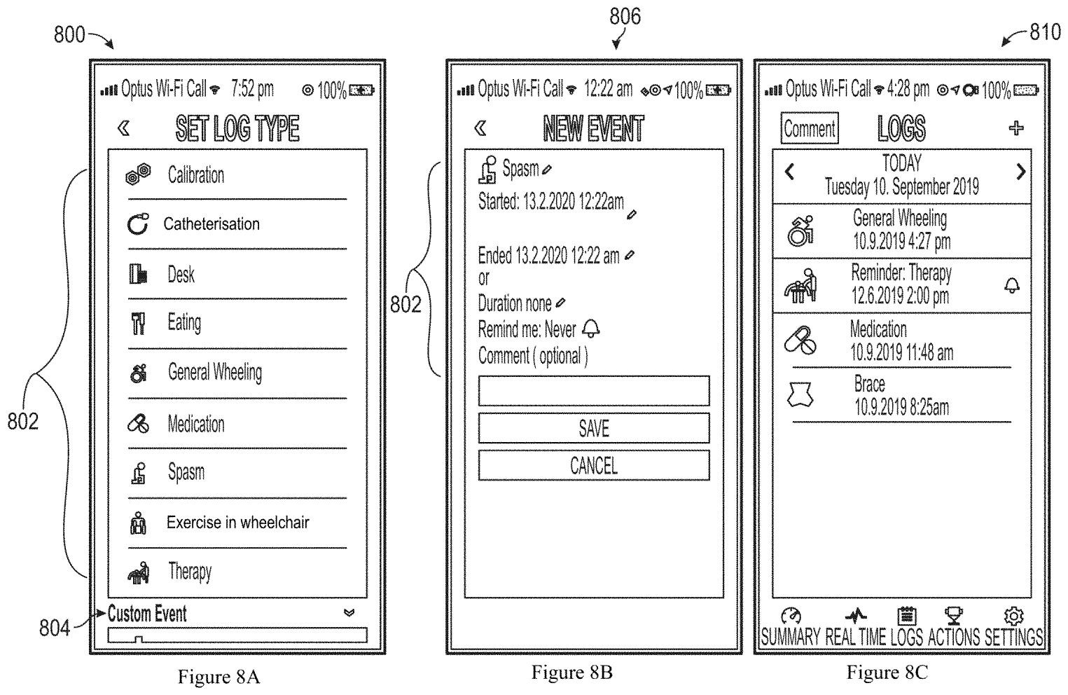

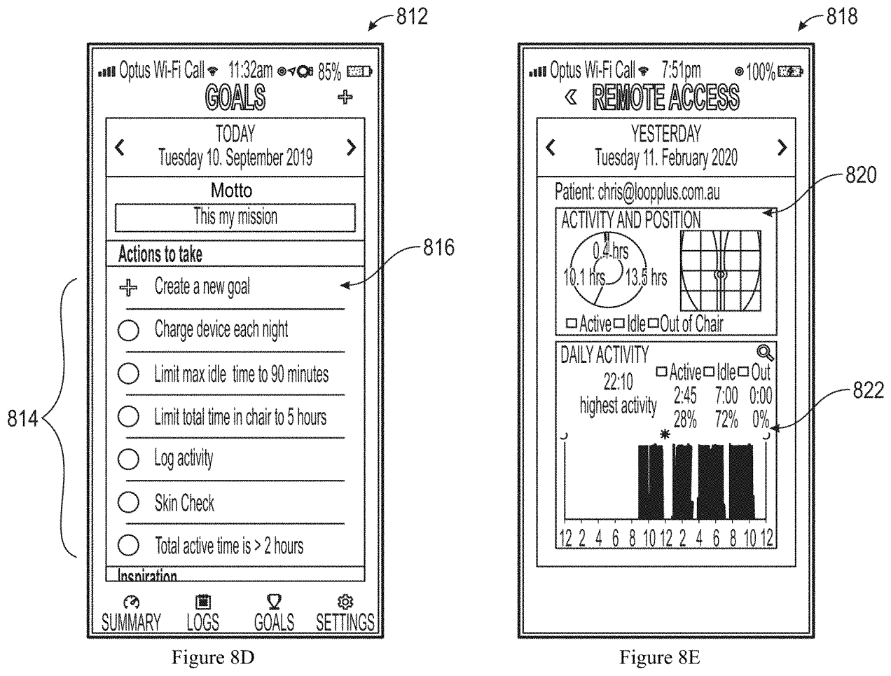

For example, referring to FIGS. 8A to 8C, the user application 712 may include various UIs that are arranged to enable the user to enter data into the user reported events module 714. For example, a UI 800 may be displayed when the user wants to log a new event. They may select from a list of predefined event types 804 that may include device calibration, catheterisation, sitting at a desk, eating, general wheeling, taking medication, experiencing a spasm, performing exercise in their wheelchair or having physical therapy. Alternatively, the user may enter a custom event 804 if their event is not provided in the list.

The user application 712 may then display UI 806 that enables the user to enter the details of the event. For example, where the event is a spasm, the user may enter the details 808 such as the start and finish time, or duration, whether to set a reminder about the event and when to set such a reminder, and the option to add any comments regarding the event. The details 808 may be entered manually by the user by typing into their mobile device, web interface or by voice activated commands. Alternatively, the details may be prefilled by the system 400 due to the system detecting the state of the user using the sensing device 100.

The user application 712 may also be configured to display to the user a summary of the events that they have logged during a 24-hour period. Referring to FIG. 8C, a UI 810 provides a list of the events that the user has logged for that day, each entry on the list including the type of event, the date and time it occurred and whether a reminder has been set in relation to that event. Such events can be filtered to show event type over a requested time period to inform progress milestones and/or clinical intervention.

In an embodiment, the user application 712 may also be configured to allow the user and/or their circle of care to set goals and monitor their progress. For example at FIG. 8D, the user application 712 may display a UI 812 that shows a list 814 of the daily goals that the user has set for themselves and the option to set a new goal 816. The system 400 may be configured to monitor said goals and notify the user and/or their circle of care when each and/or all of the goals have been met as determined by the system 400 or entered by the user.

The user application 712 may display further information to the user. For example, FIG. 8E illustrates a user interface 818 that may include graphical representations providing a real-time summary of activity and position 820 and a log of the periods of daily activity 822 as determined by the system 400.

Referring again to FIGS. 7A and 7B, the controller module 500 may be configured to process and analyse the data sets from the sensing device 100, along with other information provided by the user or gathered by additional sensors, to determine the state of the user.

Within the above described method, the step of processing the data using the controller module to identify one or more user events may include a number of different processes. One type of processing performed by the controller module 500 may include sensor fusion. As such, an embodiment of the system 400 may be provided where the user's mobile device that includes the user application 712 collects user data from the user and/or a user's circle of care. The controller module 500 is further configured to undertake data fusion processing of the data collected. The data collected may include data from any one of the plurality of sensors 104, the plurality of further sensors 738, and the user data from the user application 712, wherein based on the data fusion processing, the controller module 500 classifies user events with respect to the mobility assistance device to determine the state of the user.

For example, where the plurality of sensors 104 includes a plurality of pressure sensors 101, at least one IMU 103, and at least one strain measurement device 305, the controller model 500 may perform the process of sensor fusion to merge the data from each of the different sensors in the sensing device and the further plurality of sensors, which may include temperature, humidity and barometric pressure, to transform the data into an actionable insight including estimating and communicating health risk to enable early intervention. Risk level and the communication thereof may be further determined by the data in the user application. Various data fusion methods or algorithms may be used to undertake the data fusion including; central limit theorem, kalman filter, bayesian networks, dempster-shafer or convolutional neural network algorithms.

The system 400 takes in the data from the sensing device 100 and a further plurality of sensors 738 located on the wheelchair 202 or in the controller module 500, such as IMU sensors, temperature sensors, humidity sensors and or any of the above mentioned sensors. The control module 500 undertakes processes to determine and communicate the state of the user, including retaining the device configuration data and user event logs, data pre-processing, event detection, classification, training the neural network and alerts.

Alternatively, the above mentioned functions, processing or modules included in FIG. 7B may be performed by or located on remote terminal 704, remote cloud service 710 or the mobile device 706. For example, the remote cloud service 710 and/or remote terminal 704 may be configured to undertake data fusion or event detection 742 or may include the machine learning module 716. Moreover, the remote cloud service 710 and/or remote terminal 704 may be used to store various forms of data 744, such as user historical data, settings data, calibration data, archived raw data, and/or other user data. Further, the remote cloud service 710 and/or remote terminal 704 may also be configured to use these various data sources to undertake further analysis or processing of the data and manage any alerts that arise from that process. For example, the system 400 may also include data taken from other devices 746, such as third party wearable devices, such as smart watches, heart monitors and the like, and/or weather conditions from meteorological websites.

Referring again to the above mentioned method, the step of processing the data using the controller module 500 to identify one or more user events may further include determining an engagement state of the individual user in respect of the mobility assistance device, wherein the engagement state is one of the following:
a. Not engaged with the mobility assistance device, which means that the user is not sitting in the wheelchair. This event may be identified by comparing sensor data against a threshold set by the user during out of chair calibration. For example, a pressure reading from the plurality of pressure sensors 103 that detect a pressure above a threshold represents someone applying pressure or sitting on a pressure sensors 103, and a reading below this threshold represents someone not occupying the wheelchair. If the controller module 500 receives a new pressure reading, it will compare the new reading against threshold, wherein the event will be recorded as either the pressure sensors 103 detecting a state of “out of wheelchair” or not. In the case that all or a proportion of the sensors detecting a state of “out of wheelchair”, the controller module 500 will cease recording raw data and checking for any other events. In the case that even one or more of the pressure sensors 103 goes above this first threshold, representing a significant pressure, the controller module 500 will continue to record raw data and check for other events. That is, the readings from the pressure sensors 103 provided to the sensing device are below a not engaged force threshold.
b. Partially engaged with the mobility assistance device, which represents the user being in a partially seated position that where the body experiences pressure at a level that would still allow for blood to still perfuse in the human tissue. For example, a partially engaged position may be where the user is being partially supported by the mobility assistance device at the same time as being also supported by their legs, arms or the user's clinician. When a user is partially engaged with the mobility assistance device, the user's musculature and circulation systems experience a “relief” from the force caused by their own body weight when they are in a sitting position fully supported by the mobility assistance device. That is, the readings from the plurality of pressure sensors 103 provided to the sensing device 100 are above the not engaged threshold and below an engaged force threshold.
c. Fully engaged with the mobility assistance device, which means that the user is fully supported in a seated position by the mobility assistance device. When a user is fully engaged with the mobility assistance device, the user's musculature and circulation systems experience force from the body weight of the user. That is, the readings from the plurality of pressure sensors 103 provided to the sensing device 100 are above the engaged force threshold and below an impact force threshold.
d. Impacting with the mobility assistance device, where the user has collided with the sensing device with sufficient force to potentially cause injury. For example, where a user attempts to lift themselves out of the wheelchair 202 using their arms but collapses back into a sitting position. In this state, the readings from the plurality of pressure sensors 103 provided to the sensing device 100 are above the impact force threshold.

In a further embodiment, processing the data using the controller module 500 to identify one or more user events may include the controller module 500 classifying events and behaviours experienced by the user over a period of time. For example, processing the information from the plurality of sensors 104 to determine the presence of an event and classifying that event. The classification of the event may fall into any one of the following non-exhaustive categories; a pressure event, an impact event, an off-centre event, a body movement event, mobility assistance device event and a user activity, which are described in further detail below.

A first event described is a pressure event, which is where the user experiences force in a way that may be detrimental to their health. For example, where a user has been sitting in a wheelchair for too long and have lost circulation to their lower extremities. For example, the determination of a sustained pressure event may be performed in the following manner:
1. First a score is calculated for the rise and fall of the sustained pressure score, by:
   i. Determining a reading frequency (RF) in seconds.
   ii. Include a factor of safety (FOS) given as a percentage greater than 100. For example, the factor of safety may be equal to 150%.
   iii. Calculate a rise score (RS).

$$RS = ( \frac{seconds per hour}{desired frequency of relief in seconds} ) \times RF \times FOS$$

iv. Calculate fall score (FS).

$$FS = ( \frac{seconds per hour}{desired duration of longest relief in seconds} ) \times RF \times FOS$$

2. Next using these scores, the algorithm will start once the controller module reports a seated position on at least one sensor. Note: the algorithm may reset itself every time an out of wheelchair event is detected that lasts over a certain period of time (for example 5 minutes), or the score drops to 0 for all sensors in consideration.
3. For any and all sensors that are experiencing a “seated pressure” the algorithm will begin accumulating points at a rate specified by 1 increment of rise score per reading (charge rate).
4. This score will continue to accumulate until a sensor level drops below the “relief threshold” for:
   i. For every reading that is bellow this threshold, the calculated “fall score” from the total sustained pressure score for that sensor will be subtracted. (discharge rate)
   ii. If the sensor reading returns to a “seated” pressure, the score will continue to accumulate.

The user and/or clinician may prescribe a seating protocol specific to the individual user that enables an alert and recording of sustained pressure risk. Utilising the risk metric of sustained pressure, the protocol is set as
i. the duration of sustained seating before a relief movement is required, and
ii. the duration of relief required.
   For example, a duration of a relief may be prescribed as thirty seconds of relief for every two hours of sitting.

Further sustained pressure may be processed and visualised in real time to the user incrementally, to take account of small movements of relief. Use of the sustained pressure tracking in combination with understanding the user's care requirements, in keeping within a factor of safety, enables for determination of the risk to the user and when and where reliefs are needed. For example, measuring the effect and frequency of pressure reliefs, rather than focusing on what type of relief is actually performed (left/right/forward/back leans or lift).

Additionally, as there is no discrimination against the type of movement, the controller module 500 is able to determine any body movements that may represent an effective relief of pressure. For example, if a user shifts their weight by repositioning their legs, certain sensors will experience a relief event. Accordingly, the user's sustained pressure metric will be reduced according to the time the particular sensors readings were below a “relief threshold”. As such, the controller module 500 is able determine that a relief action is needed and alert the user accordingly.

Another event is the off-centre event, where the user is engaged or partially engaged with the mobility assistance device in way where their body weight is not evenly distributed or supported. For example, the user leaning to one side of the body, creating additional force on one side. In an embodiment, the controller module or the further computing system may also determine an off centre event by analysing the features relating to the user's pressure related position on their wheelchair. The calculations utilize raw pressure reading and the sensor's actual position on the mat (described using X, Y coordinates) to calculate centre of pressure. From this metric, further information can be determined about how the person is sitting and general information about their body's movement in the wheelchair.

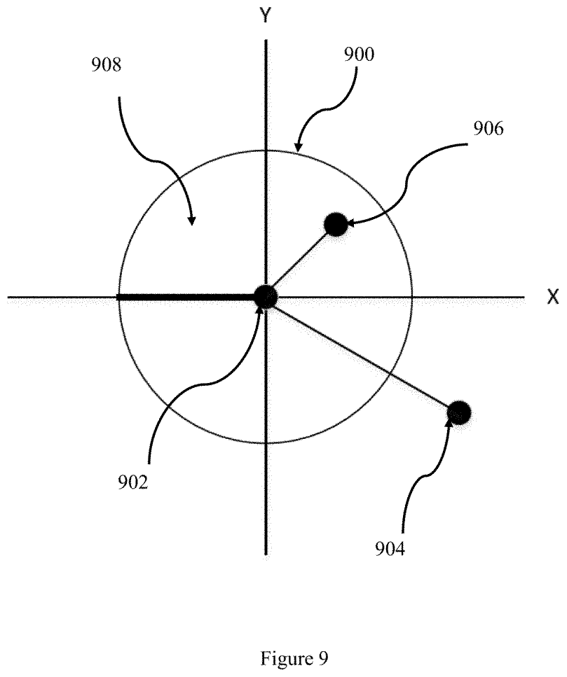

Some of the calculations used in this process are described below and with reference to FIG. 9. For example, centre of pressure (COP) is used to determine when the user has been seated off their most effective centred position. In order to perform these calculations, a “centre” point must first be established. In one embodiment, the centre can be the centre of the pad that may be described by an X and Y Cartesian coordinate system in millimetres. Further, this centre may also be described or illustrated to the user via a UI showing a similar diagram to FIG. 900 being a circle 900 that extends from a centre 902 with a radius 908 set to a threshold value. The area within this circle represents a safe centred position zone, in to which the user should aim to sit. Using the centred position zone established, the user's COP can be calculated and analysed to determine whether the user is inside or outside of their most effective centred position zone. For example, the calculated Centre of Pressure Distance from Origin (COPd) to the centre's radius 908 to determine whether the user is in one of two states; inside the centre or outside, which is illustrated by COP1 904 and COP2 906 in respectively. As in the previous event detection algorithms, every time a new pressure sensor reading is processed by the processing module this state will be checked and any change will be recorded as an event.

$$Center of Pressure in X = COPx = \frac{Σ ( ( Sensor Reading * Sensor X position ) … )}{Σ ( Sensor Readings )} ( mm )$$

$$Center of Pressure in Y = COPy \text{:} = \frac{Σ ( ( Sensor Reading * Sensor Y position ) … )}{Σ ( Sensor Readings )} ( mm ) \\ Center of Pressure Distance from Origin = COPd = \sqrt{{COPx}^{2} + {COPy}^{2}} ( mm )$$

$$Center of Pressure Velocity = COPv = \frac{( {COPd}_{i} - {COPd}_{i - 1} )}{( {time}_{i} - {time}_{i - 1} )} ( mm \text{/} sec ) for period i$$

Another event is the body movement event, which is where the controller module or the further computer may also determine how the user's pressure related position on their mobility assistance device relates to the body's actual movement in the mobility assistance device. For example, as the user will move their upper body in activities of daily living the forces will translate to the lower part of the body creating weight shifts that may support the healthy flow of blood, as described by the COP itself. Thus, by taking the difference in COP over time, the rate of change of the COP described by the Centre of Pressure Velocity (COPv) can be calculated. By calculating this value for any new filtered sensor readings, a threshold can be determined and set in accordance with the weight shifted by the state of the user's upper body movement. An example of these states can be idle, active, and highly active. Thus, as in the case of the other events, by setting individual thresholds corresponding to degree of movement in calibration and checking COPv against them, any events of changes in the user's state of body movement and position can be recorded and communicated to the user and/or the user's circle of care.

Another event is the mobility assistance device event, which is where the controller module 500 analyses the movement of the mobility assistance device itself. This information coupled with other data such as body movement may provide many additional insights on a user's behaviours and participation in activities of daily living. These will be very important in ensuring a healthy level of activity is maintained and increased independence is monitored. In this case the information derived from the further plurality of sensors 738 (such as an IMU) is used by the controller module 500 to describe acceleration and other metrics about the wheelchair's movement in up to 3 axes. As in the case of body movement, one the state of the user is classified with the fused data and user logged events to identify the wheelchair's interaction with it's physical environment such as idle, self-propelled, third party propelled, speed of movement, tilt of chair, friction of terrain. As in the other processed event, any change in state will be recorded as an event.

Another event is the environmental condition, which is where the controller module 500 determines environmental conditions that may pose health risks to the user. For example, environmental conditions often associated with pressure injuries and general skin care are temperature and RH. Such risks usually arise from ambient conditions that cause the user to sweat excessively but can also include dry and cold conditions that cause the skin to become to dry and damaged. In this case both temperature and humidity conditions may be monitored separately and have individual thresholds set for each to describe various states in consideration of their clinical risk in the user application 712. For example, temperature may simply be split into cold, normal, and hot and RH into dry, normal, and humid using two thresholds each. Alternatively, the data is first fused to produce something like a heat index and then checked against a single threshold.

As in the case of the other events readings will be checked upon receiving new data and any change in state will be recorded as an event. Furthermore, readings of temperature and humidity conditions may be combined together to give a single score indicating excessive conditions which may pose health risks to the user.

Another event is a user activity, which is where the controller module or the further computer may also determine other features of the user's movements or activities. For example, the controller module 500 may determine the number of propulsions that a user may do on a daily basis. This may be undertaken by analysing data from the further plurality of sensors 738, such as the IMU. In another example, that may require intelligently combing data from several sensors to detect particular types of reliefs, transfers, and postures. This may also extend to providing telemetry and analysis of specific sports related activities. The determination of such movements or activities may use machine learning algorithms trained against large amounts of data from different users to develop sufficiently accurate models.

As such, the above method further includes the steps of analysing the one or more user events to determine the state of the user. For example, the further terminal 704, mobile device 706 or controller module 500 may be configured to determine that the user has just attempted to lift themselves out of the wheelchair 202 and fallen back (impact event) causing the wheelchair 202 to fall over (mobility assistance device event). Alternatively, the controller 500 may determine that the user has not moved on the wheelchair 202 for a sustained period of time (pressure event) and is seated in a poor position (off-centre event) and the surrounds are hot and humid (environmental conditions). Knowing, the user's state is important in determining whether their state is detrimental to their health, which is described in further detail below.

In an embodiment, the further terminal 704, mobile device 706 or controller module 500 may be arranged to analyse the state of the user over the plurality of time periods to determine the user's risk level or risk metric. Any event that reduces the risk metric is seen to be beneficial to the user and any event that increases the risk metric is seen to be detrimental to the user. Beneficial is a term used to describe the user's state being beneficial to their health and detrimental is a term that is used to describe the user's state being detrimental to their health, by causing or contributing to the development of medical issues.

In order to determine the risk metric of each user, each user's unique characteristics may be taken into account. For example, the risk metric may include physical variables such as the user's height, weight, gender and the type and features of the wheelchair as recorded in the user application 712. The risk metric may also include the user's health history, such that if a user has a history of a certain condition or are relatively more predisposed to that condition, then it is more likely that they will develop that condition which is reflected by the user's detrimental state. In an embodiment, the user application 712 and/or controller module 500 may prompt the user in real time to alter their state in accordance with the risk metric to reduce the occurrence of the detrimental state of the user.

The risk metric includes various types of risk related to a condition or type of health issue. In each case, the user reported event or the raw data may be utilized to determine the presence of an event that may increase the risk of a user developing a health concern, and when and/or how often they occur. For example, when considering pressure events, impacts or the lack of adequate reliefs may greatly increase a user's risk of developing a pressure injury. Further, a consistently high level of RH may increase a user's risk of developing a skin condition.

In addition to these events or lack of events, user defined settings are also used to determine each user's individual risk profile. Each risk profile determines how significant each event may be in increasing an individual user's risk metric. For example, a user with frequent skin conditions with higher than average perspiration may have a higher risk of developing a skin condition after sitting in a hot environment.

Risk metric weightings indicate the probability or likelihood associated with each of the risk metrics, which are used together with the user's risk profile to define the user's overall risk. Each of the separate risk metrics are shown in the UI and be used to determine how the state of the user is recorded and when alerts are sent to a user and their circle of care (i.e. clinicians, carers and the like). A non-limiting example of some of the specific areas of risk that may be used in formulating a user's overall risk metric are set out below.
a) Pressure risk, a risk metric that may be related to the risk of developing a pressure injury that includes variables such as; pressure events, sustained pressure, time in the wheelchair, body movement, wheelchair movement, and/or transfers to and from the wheelchair (referred to as “transfers”), friction terrain, temperature, humidity and prior health history documented in the user application 712. For power wheelchair users this would include the tilt of the wheelchair.
b) Position risk, a risk metric that may be related to the risk of developing muscular skeletal issues that includes variables such as; off centre events, pressure patterns, transfers with the user's prior health history documented in the user application 712.
c) Inactivity risk, a risk metric that may be related to obesity and cardiovascular health that includes variables such as; body movement, wheelchair movement, self or third party propulsion, time in the wheelchair, transfers.
d) Shear risk, a risk metric that may be related to the risk of developing skin conditions that includes variables such as; skin integrity, sheering of the buttocks (or other areas) in relation to the wheelchair cushion, temperature, relative humidity, body movement, wheelchair movement.
e) Environmental Risk: temperature RH and other sensors used to measure ambient atmosphere may indicate the likelihood of skin related issues developing, as high heat can promote sweating that can create fungal or bacterial infections as well as how it relates to pressure injury risk.

As such, the risk metric factors in events, such as pressure events environmental conditions, and the risk profile for the particular user. By understanding their state, a user and their circle of care can track real time events and conditions relating to their body and the risk that of that leading to a detrimental state. This enables users to be proactive in changing their seating behaviours, habits and patterns to reduce the likelihood of a detrimental state. The processed data and/or analysed data may be presented to the user, and/or the user's circle of care as metrics displayed in a UI as shown in FIGS. 8C and 8E. Further, the processed data may be analysed and presented as a series of insights about the user's risk and overall activity and recommendations to reduce the user's risk.

In an embodiment, the system 400 also seeks to assist the user and their circle of care by sending alerts. These alerts aim to reduce the risk of the user experiencing health issues to encourage the user to take an action to lower the risk themselves. The alerts are communicated to the user and/or the user's circle of care to provide supportive collaborative and continuous care. In an embodiment, wherein a UI is provided to a mobile device, alerts will appear as popup notifications on the user's mobile device. Sensory alerts may also be provided via audio, visual (for example lights) or haptic feedback. Alternatively, alerts may be provided as an email, messenger app message or text message to the user and their circle of care. Further, alerts may be communicated through in home or hospital ambient computing devices such as Amazon's Alexa, Google or Apple home.

In an embodiment, the alerts may point the user to the clinician prescribed care plan or suggested actions to lower their risk. In an embodiment, an alert protocol may be informed by the user's risk level to prevent the user from being overwhelmed with notifications. It may send a single alert reporting a health risk, with details accessible in the mobile application or web interface. The detail specifies the measurement of each specified risk together with a log containing the user-logged events preceding the risk alert.

Most sensors require calibration in order to ensure accurate readings. However, in the case of the present invention, the wide variability of users and mobility assistance devices makes the calibration of the various sensors very challenging, as the hardware needs to be accurately calibrated in respect to each individual user. Further, such calibration is challenging as it should not only consider the general sensor performance (i.e. whether each sensor behaves the same each time) but also that the individual event thresholds should also be calibrated based on dynamic behaviour of the individual in each wheelchair. However, from a user experience point of view, it is preferable to significantly limit that need for recalibrating the sensors, as frequent calibration will either make the user lose interest in the sensing device and system or still use the un-calibrated product gaining inaccurate results. Therefore, the present invention includes a method for calibrating a plurality of sensors for use with a mobility assistance device, both before and after the sensor layer is sealed in the cover, the method comprising the following steps.

First, before any measurements are taken the sensors should be conditioned. In order to do this one should apply 110% or more of the sensors max force rating onto the sensor for three to five seconds. The sensor should then be allowed to rebound back to zero and should rest for another three to five seconds before the force is reapplied. This process should be repeated four to five times before the sensor can be calibrated accurately. Once conditioned, the main calibration test may follow the method outlined below.

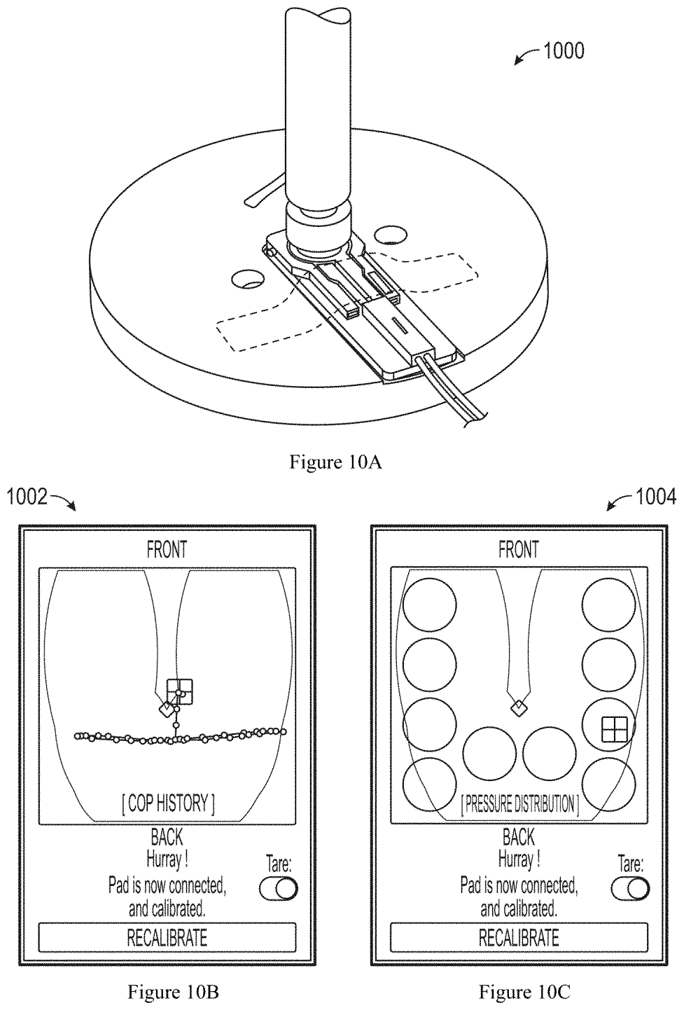

Each of the plurality of sensors 104 undergoes an initial calibration to determine the individual performance of each of the plurality of sensors 104. In an embodiment, the initial calibration seeks to test each individual sensor for quality and to ensure each sensor is normalised. The initial calibration may utilize a conventional mechanical compression force testing apparatus 1000 as shown in FIG. 10A, which applies an accurate force to the sensor and measure its response. Using this method, a puck should be placed in between the actuator and the sensors to help translate the force properly. The puck should cover 70 to 85% of the sensors sensing area to ensure all of the any force applied is translated to the sensor. The puck material should be relatively rigid. However, testing may be used to determine the optimal material to be used. In embodiments of the sensing device 100 where the outer layer 108 includes pads 110, the pads 110 replaces the puck for initial calibration.

In addition to calibrating the sensors individually, a factory calibration may also be performed where multiple sensors per pad are weighted uniformly with a known pressure on each puck, such that all the pressure sensors on a single pad may report their readings at the same time rather than testing each sensor sequentially.

Alternatively, the initial calibration may include the use of air pressure by means of a pressurized air chamber. Using the concept air pressure, a pressurized air chamber may be used to test all of the sensors at once with a homogeneous pressure. Due to the sensors design, the layers that make up the sensing device 100 may include small gaps, which are provided to allow the piezoelectric material to be compressed. As a result, these small gaps allow in air between the sensing areas. In order to accommodate a free flow of the air during compression, they are typically designed with a vent exposed to the surrounding atmosphere equalizing the pressure from the atmosphere itself therefore also removing any of its effects. Thus, in order to use the pressurized air method, the sensor's vents would need to be exposed to the atmosphere outside of the chamber allowing them to read the pressure differential.

Regardless of the method used to run the initial calibration, the initial calibration protocol should follow a similar guideline.
1. Place one third of the maximum weight rating on the sensors. Leave the weight for four to five seconds before recording the sensor reading and removing the weight. To reduce the risk of sensor drift, the time for which the weight is applied should be the same for each iteration of application.
2. Place two thirds of the intended maximum weight and again use the same interval to record and remove the weight.
3. Place the maximum calibration weight on the sensor and repeat the process.
4. Plot the data as voltage vs force and use the appropriate method to find a trend line.

As would be understood by the person skilled in the art, the protocol may include more than three iterations, that is, steps 1 to 3 may be run any number of times with the same or different weights to provide further data, where further data may also be collected to help establish corrections for sensor drift as well as dynamic sensor response.

The method for calibrating a plurality of sensors further includes undertaking user calibration to determine the cooperative performance of the plurality of sensors in respect of the user and the mobility assistance device. The user may be prompted to calibrate or recalibrate each of the plurality of sensors. In addition to the calibration of the sensors themselves for normalization purposes, the plurality of sensors may be calibrated in relation to the overall sensing device and algorithms in their final setting to account for difference in both the individual and the user's mobility assistance device. In terms of each mobility assistance device, there will be a wide variety of variability in dimensional factors. For example, in an embodiment where the mobility assistance device is a wheelchair, the wheelchair's seat size is a key variable as it determines the size of the sensing device.

Further, other variations include seat type (hard flat or hammock sling) as well as cushion (air cell, foam, gel, and hybrids). In terms of the user, variations will include dimensional differences such as overall height, hip width and appendage length. Other more important variations may include the weight of the person, level of injury and the variations in the dynamic behaviour in the wheelchair. Each variable is captured for each user in the user application 712.

Each of these variations can have very different results in terms of event recognition such as pressure reliefs, impact, position and body movement. Due to the range of variables using a standard set of parameters for all users will result in the introduction of errors. For this reason, an additional set of calibration measures may be used in conjunction with the user's variables such as weight, injury level, cushion type as captured for each user in the user application 712. Together this information assists in determining the appropriate thresholds and algorithms to maximise the accuracy of the sensing device and system in an iterative manner, by means of machine learning models and artificial intelligence to continuously improve the accuracy of event detection and risk monitoring.

Each user follows an initial protocol to calibrate the sensor device individually. In each of these steps a snapshot and/or time series data may be recorded with the plurality of sensors that may include pressure sensors and an IMU for enhanced accuracy. The following calibration steps may be repeated for any new mats, wheelchairs, cushions or seating adjustment periodically to obtain accurate results. The steps, for a mobility assistance device that is a wheelchair 202, are as follows:
1. Undertaking a user conditioning of the plurality of sensors by sitting on the sensing device when attached to the user's wheelchair for a time period of at least five to ten minutes.
2. Taking a first reading, or a plurality of readings over a period, of the user fully engaged with the wheelchair in a seated centre position for a period of ten seconds and after which the user vacates the wheelchair.
3. Taking a second reading, or a plurality of readings over a period, of the user not engaged with the wheelchair.
4. Processing the first and second readings using the controller module 500 and saving the processed first and second readings on the controller module.

From this point the protocol may vary depending on the level of injury and ability of the user. For the purposes of illustration the method is followed for an individual using a manual wheelchair and has basic control of their trunk. As such, user resumes their seat in a centred portion and the method further comprises:
1. Taking a third reading, or a plurality of readings over a period, of the user partially engaged with the mobility assistance device in a forward direction, which relieves some force applied to the back of the seat, after which the user returns to the centred position.
2. Taking a fourth reading, or a plurality of readings over a period, of the user partially engaged with the mobility assistance device in a right-sided direction, which relieves some force applied to the left side of the seat, after which the user returns to the centred position.
3. Taking a fifth reading, or a plurality of readings over a period, of the user partially engaged with the mobility assistance device in a left-sided direction, which relieves some force applied to the right side of the seat, after which the user returns to the centred position. The user may also be asked to propel the chair in a straight line if they are a manual chair for a short period.
4. Processing the third, fourth and fifth readings using the controller module and saving the processed the third, fourth and fifth readings on the controller module.

In an embodiment, the method for calibrating a plurality of sensors may further include determining whether the plurality of sensors 104 needs to be re-calibrated. As such, the controller module 500 may be programmed to include multiple automated algorithms to help maintain an appropriate calibration and may alert the user to when a new calibration may be needed. For example, the compliant materials used in such mobility assisting devices, such as foams, gels and sling type seats, have the ability to settle and deform over time. Further, the sensors themselves are based on a deformable material as described above. Due to their construction they may “wear” over time and loose sensitivity, lowering their overall dynamic range and is typically the cause for sensor drift. Drift refers to the change of the sensor value under stable conditions over time. In FIG. 10C, a UI 1004 is provided. The UI 1004 may be provided to show the sensor calibration of the pressure sensors and/or the real time pressure distribution of the user's body with respect to the mobility assistance device as detected by the plurality of sensors 104.

As such, the controller module 500 may be programmed to request that the user recalibrate the sensors 104 on a periodic basis. Alternatively, the controller module 500 may be programmed to determine when the sensors 104 need to be recalibrated. This may be undertaken by the controller module 500 communicating to the user, via a UI 1002 on the user application 712 shown in FIG. 10B, to sit in the centred position and compare the centred position against the last previous centred position, where if a sufficient change is determined, the user repeats the above described user calibration method.

Alternatively, the controller module 500 may be programmed to perform an automated self-calibration, to at least accommodate for sensor drift, wherein the controller module 500 takes a number of samples of sensor data over time when the user is in a fully engaged position as well as when they are in an non engaged position, where the samples are compared over time to determine the slope or change in pressure over time and correct for drift sensor as needed.

### Advantages

The embodiments described herein provide a novel means of determining the state of the user with respect to a mobility assistance device. In doing so, the sensing device, system and method communicates insights to users that improve awareness of their own body to make healthy choices to improve their health, motivation and independence. It also provides useful insights to clinicians and carers that form the circle of care that can be used to inform early interventions to improve quality of life, especially for those users who may find it challenging to communicate their state to another person.

The device and its installation are designed for the rigours of everyday wheelchair use. For example, where the sensing device is integrally formed into the wheelchair itself so as to replace the seat which provides value to wheelchair manufacturers, prescribing clinicians and the wheelchair user. Furthermore, as the sensing device is attached to the mobility assistance device, more accurate readings can be determined. Further, as the configuration of the plurality of sensors and the size of the sensing device can be varied, the sensing device is able to accommodate a range of users with varying levels of injury, wheelchairs from manual to power, and cushions.

Moreover, the invention provides a new method for calibrating such a sensing device in a way that minimises the number of times recalibration has to be undertaken by the user in order to improve the user experience and the accuracy and effectiveness of all measurements. The continuity of data ensures longitudinal data from the device can be used by clinicians to track the efficacy of their interventions on functional recovery and health.

The layered arrangement of the sensing device enables it to be thinly formed so that it is does not impact the user's prescribed seating plan or comfort. The layered arrangement also seeks to reduce sensor error and provide a robust sensing device that is capable of withstanding many deformations over long periods of time. Moreover, the layered arrangement is designed in such a way to be easy to manufacture and more cost effective. Furthermore, the waterproof and easy to clean arrangement and design of the sensing device, cover and protective casing protect the sensitive electrical components from water damage, being soiled, and wear and tear.

Further, the user of temporal and analog filtering, and use of interrupts significantly reduces data transmission therefore extending battery life and improving the usability of the device in everyday life. The efficiency created by the method of compressing the data with a prescribed threshold ensures the state of the user is communicated in a timely manner above all known devices. Such aspects enable right on time alerts to be issued from a mobile device or sensory feedback such as haptic, audio or visual [lights] without the need to transfer all the raw data and delays to processing the risk metric.

The processes and operational management of the device, system and methods are also configured to each individual user. The unique combination of each user's sensor positions, sensing device size, arrangements, channels, force ratings and sensitivity of the sensing device may be stored against a unique serial number in a database for reference or communication.

The new method of classifying and recording user's activities of daily living with qualitative and quantitative data to inform individual risk ensures the alerts and insights are accurate and meaningful for each user to manage a wide range of health risks. Continuous monitoring devices that employ behaviour change techniques tied to meaningful and accurate data have been clinically proven to be more motivating and efficacious in managing health risks in chronic conditions such as diabetes and asthma. Similarly, communicating the state of the wheelchair user during the activities of daily living provides a greater understanding of the beneficial and detrimental impact of their daily activities on their health. Timely feedback can support sustainable healthy habits to manage risk everyday.

Moreover, this provides a single set of outputs for use amongst the various API clients to enable the data to be consumed by services including into the heads-up display on a power wheelchair, web or app screen visualisation or may be aggregated into a clinical system for early intervention or tracking research protocols.

### Experimental Data

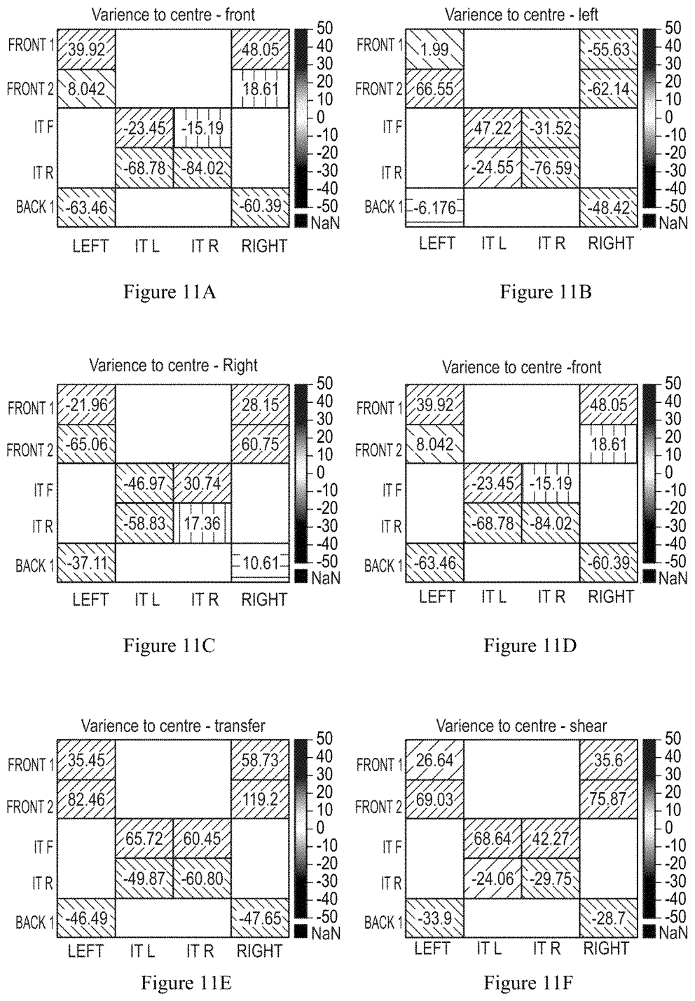

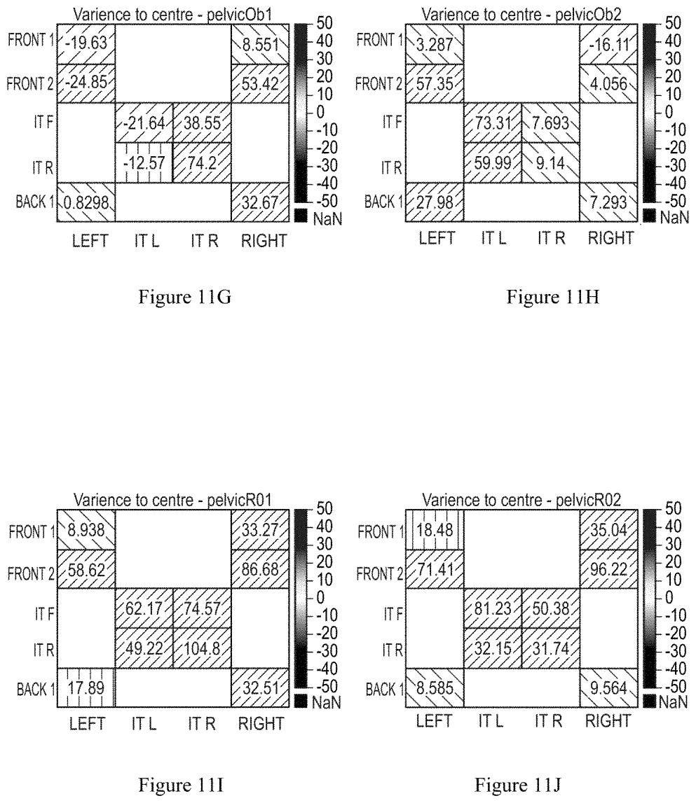

FIGS. 11A to 11J illustrate heat maps that show the sensing device 100 shown in FIG. 3C as being able to detect various seating behaviours. The variance data used in the experimental undertakings uses linearized raw sensor data. A higher value for a sensor indicates a higher pressure applied at that area of the sensing device and a lower value for a sensor indicates a lower pressure applied at that area of the sensing device.

The layout 300 denotes the locations of each of the plurality of pressure sensors 301 into rows and columns, the columns denoted left (Left), intermediate left (IT L), intermediate right (IT R) and right (Right) and the rows denoted a first front sensor row (Front 1), a second front sensor row (Front 2), a front intermediate sensor row (IT F), a rear intermediate sensor row (IT R) and a back sensor row (Back 1).

FIG. 11A shows a user in a wheelchair leaning to the front of the chair as evidenced by the higher values on the front four sensors. FIG. 11B shows the user leaning to the left as evidenced by the higher values on the left side sensors and FIG. 11C shows the user leaning to the right as evidenced by the higher values on the right side sensors. Further, it is observed that reduced pressure is provided on the alternate side to the left and right lean positions.

Further, referring to FIGS. 11D to 11F, the sensor layout at FIG. 3C is configured to distinguish between a frontal lean and similar positions. For example, FIG. 11D has a user in a frontal leaning position, whereas FIG. 11E provides a user performing a transfer and FIG. 11F shows posterior pelvic tilt which can induce shear forces as the pelvis slides down the cushion on the wheelchair. The two-by-two array of the second array 308 of pressure sensors 301 positioned in the pelvic region 312 of the sensing layer 102 shows the state of the user in their most effective centred position, forward, left and right leans and such seated orientations that demonstrate an obliquity of the pelvis. The second pair of sensors 314 ensures an accuracy in detecting other at risk seated orientations such as pelvis tilt rotation and non-ideal centre positions.

Referring to FIGS. 11G and 11H, the user is in in a position of pelvic obliquity in the right and left directions respectively. Pelvic obliquity refers to a seating position where a user is sitting and slumping so that their head shifts in the opposite direction to their torso and their body forms a c-shaped curve. For example, 11G shows higher readings on columns IT R and RIGHT, but with a with a relatively lower FRONT 1 RIGHT sensor reading. The converse is observed in respect of FIG. 11H.

Referring to FIGS. 11I and 11J, the user is in in a position of pelvic rotation in the right and left directions respectively. Pelvic obliquity refers to a seating position where a user twists one hip forward relative to the other hip. For example, 11I shows higher readings in the intermediate rows and columns, but with a with a relatively lower FRONT 1 LEFT and BACK 1 LEFT sensor reading. The converse is observed in respect of FIG. 11H.

1. A sensing device for use with a mobility assistance device, the sensing device comprising:
a sensing layer including a plurality of sensors being arranged along at least one plane,
a top outer layer and a bottom outer layer that are sealingly arranged to enclose the sensing layer,
wherein the sensing device is arranged to locate between the user and the mobility assistance device, and is configured to attach to the mobility assistance device so that the sensing device remains in the same position relative to the mobility assistance device.

2. The sensing device in accordance with claim 1, wherein the mobility assistance device is a wheelchair including a seat frame arranged to support a wheelchair seat, where the sensing device is configured to attach to the seat frame and sit on top of the wheelchair seat by means of one or more mechanical devices.

3. The sensing device in accordance with claim 1, wherein the mobility assistance device is a wheelchair including a seat frame arranged to support a wheelchair seat, where the sensing device is configured to attach to the seat frame and replace the wheelchair seat by means of one or more mechanical devices.

4. The sensing device in accordance with any one of the preceding claims, wherein the plurality of sensors includes a plurality of pressure sensors, wherein the plurality of pressure sensors are force-sensing resistors or force-sensing capacitors.

5. The sensing device in accordance with claim 4, wherein the plurality of sensors further includes at least one an inertial measurement unit and/or at least one strain measurement device.

6. The sensing device in accordance with claim 5, wherein the at least one strain measurement device is attached to a sensing layer facing-side of the top outer layer.

7. The sensing device in accordance with claim 5 or 6, wherein the at least one inertial measurement unit is arranged in the sensing layer and located proximate to the periphery of the sensing layer.

8. The sensing device in accordance with any one of claims 4 to 7, wherein the plurality of pressure sensors in the sensing layer are arranged in a first array, the first array including two columns of pressure sensors, each column of pressure sensors being symmetrical and parallel with respect to the user's sagittal axis.

9. The sensing device in accordance with claim 8, wherein the plurality of pressure sensors in the sensing layer are further arranged in a second array, wherein the second array is arranged to locate within the pelvis region and includes one or more pairs of pressure sensors, where each of the one or more pairs of pressure sensors are symmetrical with respect to the user's sagittal axis.

10. The sensing device in accordance with claim 9, wherein the second array is arranged to locate within the first array.

11. The sensing device in accordance with any one of the preceding claims, wherein the sensing layer is a flexible printed sheet, such that the plurality of sensors and the sensing layer are integrally formed.

12. A system for use with a mobility assistance device, comprising at least one sensing device including a plurality of sensors, the plurality of sensors being in communication with a controller module, wherein the at least one sensing device is arranged to locate between the mobility assistance device and a user and attach to the mobility assistance device so that the at least one sensing device remains in the same position relative to the mobility assistance device, wherein the plurality of sensors collect data that is communicated to the controller module to enable the controller module to determine a state of the user in respect of the mobility assistance device.

13. The system in accordance with claim 12, wherein the plurality of sensors includes a plurality of pressure sensors, at least one inertial measurement unit, and at least one strain measurement device.

14. The system in accordance with claim 12 or 13, wherein the controller module includes a processing module, a memory module, a communication module, an on-board sensor module, a data filter module, a power protection module, and a power access module.

15. The system in accordance with claim 14, wherein the on-board sensor module includes a plurality of further sensors selected from the group of; a temperature sensor, a relative humidity sensor, barometric pressure sensor, global positioning system sensor, a magnetometer, a three-axis accelerometer, a three-axis gyroscope, three-axis magnetometer.

16. The system in accordance with claim 15, wherein the controller module interrogates at least one of the plurality of sensors and the plurality of further sensors to obtain sensor data, wherein the controller module is configured to undertake data fusion processing on the sensor data.

17. The system in accordance with any one of claims 12 to 16, wherein the controller module is configured to be part of the sensing device.

18. The system in accordance with claims 12 to 17, wherein the controller module includes a power source in connection with the power connection module and power access module, wherein the power source includes at least one lithium battery or at least one nickel-metal hydride battery.

19. The system in accordance with claim any one of claims 12 to 18, wherein the system further includes an interface module for communicating alerts to the user.

20. The system in accordance with claim 16, wherein the system is configured to communicate via the communication module with a mobile device under the control of a user, the mobile device including a user application that collects user data from the user and/or a user's circle of care, wherein the controller module is further configured to undertake the data fusion processing of the sensor data collected by any one of the plurality of sensors, the plurality of further sensors, and the user data, wherein based on the data fusion processing, the controller module classifies user events with respect to the mobility assistance device to determine the state of the user.

21. The system in accordance with claim 20, wherein the user application further displays to the user any information relating to the classification of the user events with respect to the mobility assistance device and the events taken or experienced by the user whilst engaged with mobility and assistance device.

22. A method for determining the state of a user in respect of a mobility assistance device using the system in accordance with any one of claims 12 to 21, wherein the method comprising the steps of:
communicating data from the plurality of sensors to the controller module,
processing the data using the controller module to identify one or more user events,
analysing the one or more user events to determine a state of the user, and
analysing the state of the user over a plurality of time periods to determine the user's risk metric.

23. The method in accordance with claim 22, wherein the method further comprises the step of prompting the user and/or a user's circle of care to alter the user's state by means of an alert if the risk metric reaches a predetermined risk limit by means of a user application.

24. The method in accordance with claim 23, wherein the method further comprises the step of alerting the user and/or the user's circle of care that they have reached a goal by means of a user application.

25. A method for calibrating a plurality of sensors in a sensing device in communication with a controller module, where the sensing device and the controller module are for use with a mobility assistance device, the method comprising the steps of:
undertaking an initial conditioning of each of the plurality of sensors,
undertaking an initial calibration to determine the individual performance of each of the plurality of sensors, and
undertaking a user calibration to determine the cooperative performance of the plurality of sensors in respect of a user and the mobility assistance device.

26. The method in accordance with claim 25, wherein the step of undertaking user calibration further comprises the steps of:
undertaking a user conditioning of the plurality of sensors,
taking a first reading of the user fully engaged with the mobility assistance device,
taking a second reading of the user not engaged with the mobility assistance device, and
processing the first and second readings using the controller module and saving the processed first and second readings on the controller module.

27. The method in accordance with claim 26, wherein the step of undertaking user calibration further comprises the steps of:
taking a third reading of the user partially engaged with the mobility assistance device in a forward direction,
taking a fourth reading of the user partially engaged with the mobility assistance device in a right-sided direction,
taking a fifth reading of the user partially engaged with the mobility assistance device in a left-sided direction, and
processing the third, fourth and fifth readings using the controller module and saving the processed third, fourth and fifth readings on the controller module.

28. The method in accordance with claim 26 or 27, wherein the method further comprises the step of determining whether the plurality of sensors needs to be re-calibrated.
